(19) United States
(12) Patent Application Publication
(10) Pub. No.: US 2026/0108168 A1
(43) Pub. Date: Apr. 23, 2026

(54) SYSTEMS AND METHODS FOR ARTIFICIAL INTELLIGENCE ASSISTED MEDICAL DEVICE LOCALIZATION

(71) Applicant: Anumana, Inc., Cambridge, MA (US)

(72) Inventors: Deepak Anand, Doddanekundi (IN); Yogisha Heggadahalli Jayendra, Bengaluru (IN); Karthik K. Bharadwaj, Bengaluru (IN); Sughosh Indurkar, Bangalore (IN); Rakesh Barve, Bengaluru (IN); Animesh Agarwal, San Mateo, CA (US)

(73) Assignee: Anumana, Inc., Cambridge, MA (US)

(21) Appl. No.: 18/920,065

(22) Filed: Oct. 18, 2024

(51) Int. Cl. A61B 5/06; A61B 34/20

(52) U.S. Cl. CPC A61B 5/065; A61B 34/20

(57) ABSTRACT

A system for artificial intelligence assisted medical device localization, wherein the system includes at least a processor; a catheter communicatively connected to the at least a processor, and a memory containing instructions configuring the at least a processor to localize the catheter. The catheter may include one or more electrodes configured to detect a plurality of potential signals and at least a magnetic sensor configured to detect position data. Localizing the catheter may include receiving position data from the at least a magnetic sensor, receiving a plurality of potential signals from the one or more electrodes, wherein the plurality of potential signals includes at least a reference electrode datum and at least a query electrode datum, instantiating a localization machine-learning model, inputting the position data and the plurality of potential signals, and determining, using the localization machine-learning model, a location of the one or more query electrodes.

### FIELD OF THE INVENTION

The present invention generally relates to the field of localization of internal medical devices. In particular, the present invention is directed to systems and methods for artificial intelligence assisted medical device localization.

### BACKGROUND

Cardiac mapping systems are advanced technologies used to visualize and analyze the heart's electrical activity in detail. These systems play a crucial role in diagnosing and treating cardiac arrhythmias, conditions where the heart's electrical signals are abnormal. By capturing and displaying real-time data on the heart's electrical conduction pathways, cardiac mapping systems allow clinicians to pinpoint the precise locations of electrical disturbances within the heart. This information is essential for guiding interventions such as catheter ablation, a procedure that targets and eliminates the sources of arrhythmias. Cardiac mapping systems have the ability to provide high-resolution, three-dimensional maps of the heart's electrical activity.

### SUMMARY OF THE DISCLOSURE

In an aspect the present disclosure illustrates exemplary systems for AI assisted medical device localization. Exemplary systems include at least a processor, a catheter communicatively connected to the at least a processor, wherein the catheter includes one or more electrodes configured to detect a plurality of potential signals and at least a magnetic sensor configured to detect position data, and a memory communicatively connected to the at least a processor. The memory includes instructions configuring the at least a processor to receive position data from the at least a magnetic sensor, receive a plurality of potential signals from the one or more electrodes, wherein the plurality of potential signals includes at least a reference electrode datum and at least a query electrode datum, instantiate a machine-learning model, input the position data and the plurality of potential signals, and determine, using the machine-learning model, a location of the one or more query electrodes.

In another aspect the present disclosure illustrates exemplary methods for AI assisted medical device localization. Exemplary methods include receiving position data from the at least a magnetic sensor, receiving a plurality of potential signals from the one or more electrodes, wherein the plurality of potential signals includes at least a reference electrode datum and at least a query electrode datum, instantiating a machine-learning model, inputting the position data and the plurality of potential signals, and determining, using the machine-learning model, a location of the one or more query electrodes.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of a particular implementation of a system for AI-assisted medical device localization;

FIG. 2A is a diagram illustrating an exemplary embodiment of a catheter with electrodes in a grid orientation;

FIG. 2B is a diagram illustrating an exemplary embodiment of a catheter with electrodes in a straight-line orientation;

FIG. 3 is a diagram of a particular implementation of a system for AI-assisted medical device localization, specifically illustrating an exemplary embodiment of the at least one or more sensors of a medical device using one or more rigid electrodes as a reference electrode;

FIG. 4 is a diagram of a particular implementation of a system for AI-assisted medical device localization, specifically illustrating an exemplary embodiment of the at least one or more sensors of a medical device using one or more non-rigid electrodes as a reference electrode;

FIG. 5 is a block diagram of an exemplary machine-learning process;

FIG. 6 is a diagram of an exemplary embodiment of a neural network;

FIG. 7 is a diagram of an exemplary embodiment of a node of a neural network;

FIG. 8 is a flow diagram of an exemplary embodiment of a software flow for various modules;

FIG. 9 illustrates an exemplary model architecture;

FIG. 10 illustrated is a flow diagram of a particular implementation of various steps of a method of AI-assisted medical device localization; and

FIG. 11 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for AI assisted medical device localization. In an embodiment, systems and methods of AI assisted medical device localization determine a location of one or more query electrodes in relation to one or more reference electrodes and at least a magnetic sensor.

Aspects of the present disclosure can be used to estimate in real-time the location and/or orientation of one or more electrodes. Aspects of the present disclosure can also be used to implement systems and methods for AI assisted medical device localization wherein non-identical electrodes are present. This is so, at least in part, because the machine-learning model may be trained to convert a first electrode type having a first electrode type characteristic into a first electrode type having a second electrode type characteristic. Systems and methods herein may then be implemented to determine the location of one or more electrodes based on this transformation.

Aspects of the present disclosure allow for AI assisted medical device localization. Exemplary embodiments illustrating aspects of the present disclosure are described below in the context of several specific examples.

During electrophysiological (EP) procedures cardiologists may insert catheters with electrodes into the heart cavities and/or on the heart's surfaces. Determining the position and orientation of a medical device, such as a catheter, in a human body may be accomplished through various systems. One such system is known as an electrical impedance-based positioning system. Generally, electrical impedance-based positioning systems include one or more pairs of body surface electrodes, a reference sensor, and one or more sensors attached to the medical device. The system may then determine the position and orientation of the medical device by applying a current across pairs of electrodes, measuring respective voltages induced at the medical device sensors, and then processing the measured voltages. Alternatively, one may implement a magnetic field-based positioning system. Generally, magnetic field-based positioning systems include one or more magnetic field generators attached to or placed near the patient bed or other component of the operating environment, and one or more magnetic field detection coils coupled with a medical device. As an alternative, the field generators may be coupled with a medical device, and the detection coils may be attached to or placed near a component of the operating environment. Using the magnetic field produced by the generators and signals produced by the detection coils, the system may process the signals to produce one or more position and orientation readings associated with the coils. Unlike an electrical impedance-based system, where the coordinate system is relative to the patient, a magnetic field-based system has a coordinate system that is independent of the patient.

Both electrical impedance-based positioning systems and magnetic field-based positioning systems have their advantages. For example, electrical impedance-based positioning systems provide the ability to simultaneously locate a relatively large number of sensors on multiple medical devices. However, because electrical impedance-based positioning systems employ electrical current flow in the human body, such systems may be subject to electrical interference and are thus less precise. As a result, geometries and representations may appear distorted relative to actual images of subject regions of interest. On the other hand, magnetic field-based positioning systems are not dependent on characteristics of the patient's anatomy and typically provide improved accuracy in comparison. However, magnetic field-based positioning systems are generally limited to tracking relatively fewer sensors in comparison to electrical impedance-based positioning systems and are typically costlier.

Efforts to combine the advantages while limiting the disadvantages of each of these systems have been made. These efforts, however, have largely failed to seamlessly integrate the two systems. The following disclosure discusses an improvement on the integration of these systems with assistance from artificial intelligence (AI).

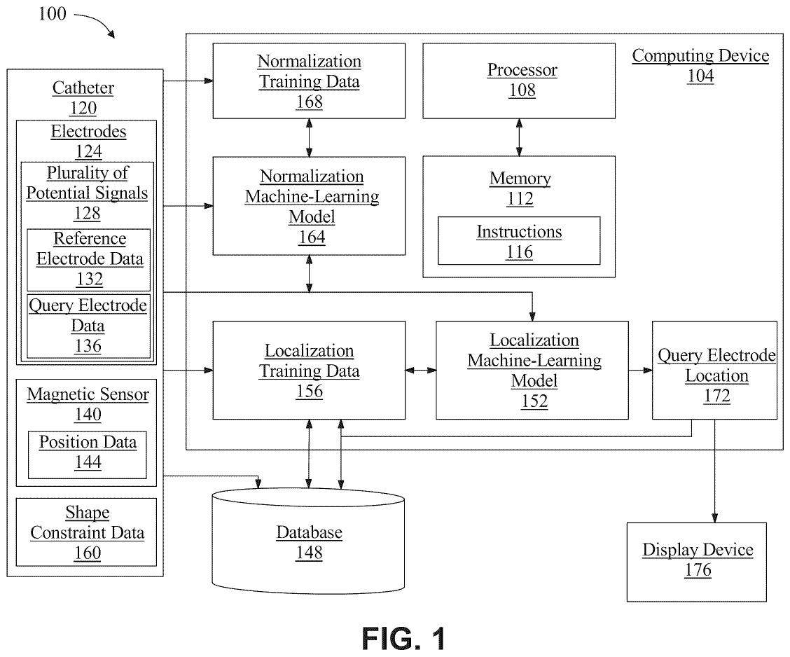

Referring now to FIG. 1, an exemplary embodiment of a system for AI assisted medical device localization is illustrated. System 100 may include at least a processor 108, a catheter 120 communicatively connected to at least a processor 108, and a memory 112 communicatively connected to at least a processor 108. Catheter 120 may include one or more electrodes 124 configured to a plurality of potential signals 128 and at least a magnetic sensor 140 configured to detect position data 144. Further, memory 112 may contain instructions 116 configuring at least a processor 108 to receive position data 144 from at least a magnetic sensor 140, receive plurality of potential signals 128 from one or more electrodes 124, instantiate a localization machine-learning model 152, input position data 144 and plurality of potential signals 128, and determine, using localization machine-learning model 152, a location 172 of one or more query electrodes. Plurality of potential signals 128 may include at least a reference electrode datum 132 and at least a query electrode datum 136. Furthermore, in some embodiments, system 100 may further be configured to display, at a display device 176, location 172 of one or more query electrodes. Any display device, as described in detail below, may display location 172 of one or more query electrodes and any associated data therewithin.

With further reference to FIG. 1, system 100 includes a computing device 104. Further, computing device 104 includes processor 108 communicatively connected to memory 112. As used in this disclosure, “communicatively connected” means connected by way of a connection, attachment or linkage between two or more relata which allows for reception and/or transmittance of information therebetween. For example, and without limitation, this connection may be wired or wireless, direct or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio and microwave data and/or signals, combinations thereof, and the like, among others. A communicative connection may be achieved, for example and without limitation, through wired or wireless electronic, digital or analog, communication, either directly or by way of one or more intervening devices or components. Further, communicative connection may include electrically coupling or connecting at least an output of one device, component, or circuit to at least an input of another device, component, or circuit. For example, and without limitation, via a bus or other facility for intercommunication between elements of a computing device. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Further referring to FIG. 1, Computing device 104 may include any computing device as described in this disclosure, including without limitation a microcontroller, microprocessor, digital signal processor (DSP) and/or system on a chip (SoC) as described in this disclosure. Computing device 104 may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented, as a non-limiting example, using a “shared nothing” architecture.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, computing device 104 may perform determinations, classification, and/or analysis steps, methods, processes, or the like as described in this disclosure using machine-learning processes. For the purposes of this disclosure, a “machine-learning process” is a process that automatedly uses a body of data known as “training data” and/or a “training set” (which is described further below in this disclosure) to generate an algorithm that will be performed by a processor/module to produce outputs given data provided as inputs; this is in contrast to a non-machine-learning software program where the commands to be executed are determined in advance by a user and written in a programming language. Machine-learning processes may utilize supervised, unsupervised, lazy-learning processes and/or neural networks, as described further below.

Still referring to FIG. 1, in an embodiment, catheter 120 may include one or more electrodes 124 and at least a magnetic sensor 140. As used throughout this disclosure, “catheter” refers to a flexible instrument configured to be inserted through an opening into a body cavity. Exemplary embodiments of catheter 120 may include Tacticath catheter and/or HDGrid catheter. Localization machine-learning model 152, discussed in further detail below, may be trained on any embodiment of catheter 120 as mentioned herein and/or any embodiment of catheter 120 within use in the field. This may allow for increased accuracy and efficiency of localization and a decrease in costs associated with the need for specific embodiments of catheter 120. This is so, at least in part, due to the nature of system 100's ability to scale different electrode types to a singular electrode type having a matching electrode characteristic. Said ability is further discussed below.

In further reference to FIG. 1, one or more electrodes 124 may be configured to detect a plurality of potential signals 128. As used throughout this disclosure, “electrode” and/or “electrodes” refer to a conductor through which electricity may enter or leave an object, substance and/or region. In an embodiment, one or more electrodes 124 may include electrodes of the same variety and/or electrodes of different varieties. For example, one or more electrodes 124 may include reference electrodes, working electrodes, counter electrodes, biosensors, bioelectrodes, electrochemical sensors, electrolytic electrodes, and/or electrodes of varying size, material, shape, and/or electrical characteristics. As used throughout this disclosure, a “potential signal” is a signal conveying the electrical potential difference between an electrode and a reference electrode. Plurality of potential signals 128 may include voltage signals, current signals, potential signals, resistance signals, impedance signals, electrochemical signals, bioelectrical signals, magnetoelectric signals, and/or the like. Plurality of potential signals 128 may include signal characteristics such as amplitude, frequency, and/or phase. Furthermore, plurality of potential signals 128 may include at least a reference electrode datum 132 and at least a query electrode datum 136, wherein the at least a datum encompasses the embodiments of data as discussed in relation to plurality of potential signals 128 and its characteristics. Throughout this disclosure, “reference electrode” and “query electrode” may be used in discussion of one or more applications of system 100; these are in reference and connection with plurality of potential signals 128 as described above and throughout this disclosure.

Continuing to reference FIG. 1, at least a magnetic sensor 140 may be configured to detect position data 144. A “magnetic sensor” is a device that detects and measures magnetic fields. Such sensors may determine the strength, direction and/or in some embodiments the rate of change of a magnetic field. Exemplary embodiments of magnetic sensors 140 may include magnetoresistive sensors, hall effect sensors, fluxgate sensors, magnetic resonance imaging (MRI)-compatible sensors, and/or the like. As used throughout this disclosure, “position data” refers to the specific location and orientation of the catheter. Position data 144 may include spatial coordinates, orientation, distance, alignment, and/or the like. Further, spatial coordinates may include x, y, z coordinates in three-dimensional space and/or polar coordinates for spherical or cylindrical setups. Orientation may include the angle and/or positioning of the electrode in relation to other components or the medium with which it is interacting with. For example, and without limitation, the angle of inclination or rotation relative to a reference plane. Further, distance may be measured from a fixed point or other reference electrodes. For example, the distance from a reference electrode and/or the distance from the magnetic sensor 140.

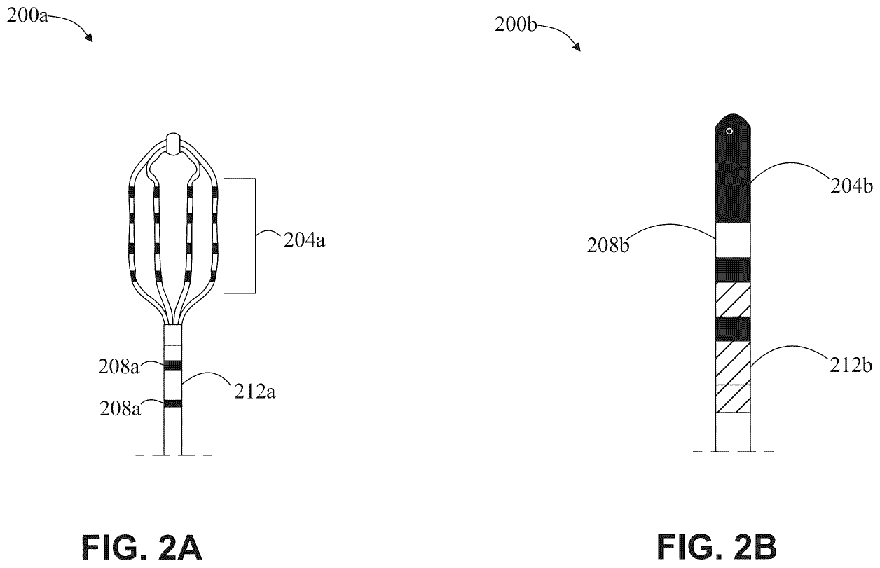

In further reference to FIG. 1, in an embodiment, one or more electrodes 124 may include one or more rigid electrodes and one or more flexible electrodes. Rigid electrodes may yield at least a reference electrode datum 132. Whereas flexible electrodes may yield at least a query electrode datum 136. As used throughout this disclosure, a “rigid electrode” is a type of electrode that maintains a fixed, inflexible shape during its operation. Unlike flexible or deformable electrodes, rigid electrodes are designed to stay in constant position and structure. For example, a rigid electrode may include metal electrodes, carbon-based electrodes, ceramic electrodes, conductive polymers, and/or reference electrodes. Each of these examples has its own advantages and/or disadvantages and may be chosen based on its specific application. As used throughout this disclosure, a “flexible electrode” is an electrode designed to bend, stretch, and/or conform to various surfaces without losing its functionality. For example, flexible electrodes may include conductive polymers, metal mesh or foil electrodes, carbon-based flexible electrodes, hydrogels, and/or stretchable elastomers with conductive fillers. These electrodes may be used in place of and/or in combination with rigid electrodes for certain applications. Flexible electrodes may adapt to different shapes and movements, making them ideal for dynamic and/or complex environments. Advantages of flexible electrodes may include flexibility, conformability, durability, and conductivity. In one or more embodiments, system 100 implements catheter 120 including both one or more rigid electrodes and/or one or more flexible electrodes. In some embodiments, one or more electrodes 124 may be positioned in a grid orientation. In such an embodiment, one or more electrodes 124 may be located in a grid, wherein each electrode is 1 mm in length and spaced 3 mm apart from one another. Additionally, in such an embodiment there may be one or more shaft electrodes located on either side of magnetic sensor 140. For example, and without limitation, like the Tacticath catheter. For a more detailed description of such an embodiment, please refer to FIG. 2A. Alternatively, in some embodiments, one or more electrodes may be positioned in a straight-line orientation. For example, and without limitation, like the HDGrid 18-electrode catheter with two types of electrodes. For a more detailed description of such an embodiment, please refer to FIG. 2B.

With continued reference to FIG. 1, a “reference electrode” is an electrode that serves as a reference relative to a query electrode. A “query electrode,” is an electrode that is being evaluated.

Continuing to refer to FIG. 1, a “reference electrode datum” is a datum collected from a reference electrode. Associated data may include electrical potential (voltage), current, concentration, stable and known potential, against which other electrodes' potentials may be measured, electrical activity of the heart, and/or the like. At least a reference electrode datum 132 may include one or a plurality of reference data corresponding to a reference electrode. Throughout this disclosure, “query electrode datum” is a datum collected from a query electrode. For example, in some embodiments, query electrode datum 136 may include electrical potentials, current, concentration, electrical activity of the heart, and/or the like. At least a query electrode datum 136 may include one or a plurality of query data corresponding to a query electrode. In some embodiments, system 100 may determine the location 172 of one or more query electrodes from the electrical potential of at least a query electrode datum 136 in combination with position data 144 from at least a magnetic sensor 140 and a known location of the reference electrode. Further, in such an embodiment, magnetic sensor 140 may be in a fixed position within 30 mm of one or more electrodes. The assumption of a 30 mm radius is fair for the dimensions of a typical heart chamber.

With continued reference to FIG. 1, in one or more embodiments, one or more machine-learning models may be used to perform a certain function or functions of system 100, such as determining a location 172 of one or more query electrodes and normalizing non-identical electrode types in order to standardize the electrode characteristics, as described in detail below. Processor 108 may use a machine-learning module to implement one or more algorithms as described herein or generate one or more machine-learning models, such as normalization machine-learning model 164 and/or localization machine-learning model 152, as described below. However, machine-learning module is exemplary and may not be necessary to generate one or more machine-learning models and perform any machine-learning described herein. In one or more embodiments, one or more machine-learning models may be generated using training data. Training data may include inputs and corresponding predetermined outputs so that machine-learning model may use correlations between the provided exemplary inputs and outputs to develop an algorithm and/or relationship that then allows the machine-learning model to determine its own outputs for inputs. Training data may contain correlations that a machine-learning process may use to model relationships between two or more categories of data elements. Exemplary inputs and outputs may come from database 148 or be provided by a user. In one or more embodiments, machine-learning module may obtain training data by querying communicatively connected database 148 that includes past inputs and outputs. Training data may include inputs from various types of databases 148, resources, and/or user inputs and outputs correlated to each of those inputs, so that machine-learning model may determine an output. Correlations may indicate causative and/or predictive links between data, which may be modeled as relationships, such as mathematical relationships, by machine-learning models, as described in further detail below. In one or more embodiments, training data may be formatted and/or organized by categories of data elements by, for example, associating data elements with one or more descriptors corresponding to categories of data elements. As a nonlimiting example, training data may include data entered in standardized forms by persons or processes, such that entry of a given data element in a given field in a form may be mapped to one or more descriptors of categories. Elements in training data may be linked to descriptors of categories by tags, tokens, or other data elements. In one or more embodiments, training data may include previous outputs such that one or more machine-learning models may iteratively produce outputs.

Still referring to FIG. 1, one or more trained machine-learning models may be evaluated. In an embodiment evaluation of one or more trained machine-learning models may include a Point-Wise evaluation, which may further include RMSE error, cumulative plot of the Euclidean error with cutoff at 90 percentile. RMSE error may be determine by calculating the root mean square of the pointwise error across x, y, and z, cumulative plot of the Euclidean error with a cutoff at 90 percentile may be determined by calculating the Euclidean error at 90% of the point population, which is ascending sorted based on the Euclidean error. In yet another embodiment, one or more trained machine-learning models may be evaluated on mesh-based metrics. A mesh-based metrics evaluation may include mean error metrics, mean square metrics, and Hausdorff metrics. The mean square metric returns the Mean Square Error (MSE) between the two sets of vertices by considering each triangle on the source object and its closest pints in the target object. The mean error metric is similar to MSE with minute differences. The main differences between mean error and mean square error lie in the error calculation steps. For mean square error, the individual distances are squared but not square-rooted, and the final error is the square root of the cumulative error divided by the total area. For the mean error metric, the individual distances are squared and the square-rooted, and the final error is simply the cumulative error divided by the total area. Hausdorff metric may include distance and is used to measure the extent to which tow subsets of a metric space differ. In simpler terms, it quantifies how far two shapes are form each other, which can be particularly useful in applications like shape matching.

In further reference to FIG. 1, system 100 may be configured to instantiate localization machine-learning model 152, input position data 144 and plurality of potential signals 128, and determine, using localization machine-learning model 152, location 172 of one or more query electrodes. In one or more embodiments, determining location 172 of one or more query electrodes may include iteratively determining sets of locations of a plurality of query electrodes using a plurality of reference electrodes. Such an embodiment may operate under the following assumptions: (1) presence of at least a magnetic sensor 140 providing real-time location and orientation; (2) one or more electrodes 124 rigidly located with respect to magnetic sensor 140 providing a fixed reference electrode and a query electrode; and (3) a transformation, such that overall, the fixed reference electrode may be compared to the query electrode. In one or more embodiments, the query electrode may be a different electrode type in comparison to the reference electrode. A fixed reference electrode, may, in some embodiments be represented as such-Fixed Δ_{ref}: =[MSₓ−V_{ref}−x location, MS_{Y}−V_{ref}−y location, MS_{z}−V_{ref}−z location]. Additionally, a query electrode, may, in some embodiments be represented as such—Δ_{query}=[MSₓ−V_{query}−x location, MS_{Y}−V_{query}−y location, MS_{z}−V_{query}−z location]. The following formulation is such that one has magnetic sensor 140 location, a known location of V_{ref}, and V_{query} at an unknown location, but within a sphere of 30 mm radius: [symbol] ([MS, V_{ref}, Δ_{ref}, V_{query}]) →>Δ_{query}. In this embodiment, the formulation requires voltage-location pairs for each electrode to make many V_{ref}, and V_{query} pairs. Localization machine-learning model 152 may be trained on exemplary localization training data 156, including inputs such as, and without limitation plurality of potential signals 128, magnetic sensor 140 location data, exemplary reference electrode voltage and position data 144, and query electrode voltage data correlated with exemplary outputs such as, without limitation query electrode position data 144. The reference electrode may be either flexible and/or rigid. The query electrode may be either flexible and/or rigid.

With continued reference to FIG. 1, in an embodiment where the query electrode is a different electrode type than the reference electrode, a transformation may be necessary. Transformation may be referred to as conversion, normalization and/or normalizing. Transformation may include time stamp matching for different data streams, including data streams from non-identical electrodes. For example, and without limitation the magnetic data stream from catheter 120, which may include any data as described throughout this disclosure, may provide 33 samples per second from magnetic sensor 140 and 10 windows of signals per second from electrodes 124. To pair data across the magnetic and the electrical/impedance measurements, the slower data is matched to the nearest record of the faster record. In an embodiment this may be modeled by the following steps: (1) for every t_{DAQ} a difference t_{diff} is computed between the last 33 tₘ magnetic timestamps; (2) pick the tₘ with the least difference t_{diff}; (3) after matching in the above step, the worst-case t_{diff} is 18 ms and the tₘ magnetic time stamp is retained for further processing downline. A typical matched row in the collected data in magnetic and impedance hardware setup for a catheter may present as such: [tₘ, MSₓ, MS_{y}, MS_{z}, Q₀, Q₁, Q₂, Q₃, V₁^{f1}, V₁^{f2}, . . . , V₁^{fj}, . . . V₁^{fm}, V₂^{f1}, V₂^{f2}, . . . , V₂^{fj}, . . . , V₂^{fm}, . . . Vᵢ^{f1}, Vᵢ^{f2}, . . . , Vᵢ^{fj}, . . . Vᵢ^{fm}, . . . , Vₙ^{f1}, Vₙ^{f2}, . . . , Vₙ^{fj}, . . . Vₙ^{fm}]. Wherein, tₘ is magnetic acquisition time (clock time); MSₓ is magnetic sensor x location of the catheter (mm); MS_{y} is magnetic sensor y location of the catheter (mm); MS_{z} is magnetic sensor z location of the catheter (mm); Q₀, Q₁, Q₂, Q₃ are quaternion coefficient providing orientation; and Vᵢ^{fj} is FFT amplitude (volts) of the j^{th} electrode (out of total n electrodes) with respect to the j^{th} patch frequencies (out of total m patch frequencies). This may be undergone using a machine-learning model, such as normalization machine-learning model 164 as described below.

Continuing to reference FIG. 1, In an embodiment, wherein one or more electrodes are non-identical, memory 112 may further include instructions configuring processor 108 to scale plurality of potential signals 128 of the one or more non-identical electrodes. Normalizing plurality of potential signals 128 of the one or more non-identical electrodes may include instantiating a normalization machine-learning model 164, receiving a first type of electrode data having a first type of electrode characteristic, receiving a second type of electrode data having a second type of electrode characteristic, and converting, using normalization machine-learning model 164, the first type of electrode data having a first type of electrode characteristic into a first type of electrode data having a second type of electrode characteristic. The output of normalization machine-learning model 164 may directly be transferred to the next step and/or be displayed at display device 172. As used throughout this disclosure, a “display device” is any output device configured to show or display information in a visual form. For example, and without limitation, display device 176 may include monitors, touchscreens, Liquid Crystal Display (LCD), projectors, graphic panels, Cathode Ray Tube (CRT), Organic light-emitting diode (OLED) displays, and/or the like. Instantiation of normalization machine-learning model 164 may include generating normalization training data 168, training the normalization machine-learning model 164, and determining, using the normalization machine-learning model 164, scaled voltages of the one or more non-identical electrodes. In one or more embodiments, normalization training data 168 includes exemplary inputs such as, but without limitation, voltage pairs measured using the one or more non-identical electrodes at a same location correlated with exemplary scaled voltages. As used throughout this disclosure, “same location” is a location that is within +/−2% of a given location. Training of the model may take place at processor 108 and/or remotely. Additionally, outputs of normalization machine-learning model 164 may be iteratively reused as new normalization training data 168 to update normalization machine-learning model 164. Updates to normalization machine-learning model 164 may occur at processor 108 and/or remotely. In a nonlimiting example, normalization machine-learning model 164 described herein may be consistent with any normalization machine-learning model 164 disclosed in U.S. patent application Ser. No. 18/911,857 (attorney docket number 1518-185USU1), filed on Oct. 10, 2024, and entitled “APPARATUS AND METHOD FOR DETERMINING A NORMALIZED VOLTAGE ACROSS NON-IDENTICAL ELECTRODES” the entirety of which is incorporated herein by reference.

Continuing to reference FIG. 1, in some embodiments, a matched row in the collected data, including plurality of potential signals 128 and position data 144, in system 100 may include: [tₘ, MSₓ, MS_{y}, MS_{z}, Q₀, Q₁, Q₂, Q₃, V₁^{f1}, V₁^{f2}, . . . , V₁^{fj}, . . . V₁^{fm}, V₂^{f1}, V₂^{f2}, . . . , V₂^{fj}, . . . , V₂^{fm}, . . . , Vᵢ^{f1}, Vᵢ^{f2}, . . . , Vᵢ^{fj}, . . . Vᵢ^{fm}, . . . , Vₙ^{f1}, Vₙ^{f2}, . . . , Vₙ^{fj}, . . . Vₙ^{fm}]. Where, “tₘ” is magnetic acquisition time (clock time); “MSₓ” is Magnetic sensor 140 x location of catheter 120 (mm); “MS_{y}” is Magnetic sensor 140 y location of catheter 120 (mm); “MS_{z}” is Magnetic sensor 140 z location of catheter 120 (mm); “Q₀, Q₁, Q₂, Q₃” is Quaternion coefficient providing orientation; and “Vᵢ^{fj}” is FFT amplitude (volts) of the i^{th} electrode (out of total n electrodes) with respect to the j^{th} patch frequencies (out of total m patch frequencies).

With further reference to FIG. 1, in an embodiment, localization training data 156 may be generated from collected data. As used throughout this disclosure, “collected data” is data collected from one or more electrodes 124 and at least a magnetic sensor 140. For example, from the given data, [[tₘ, MSₓ, MS_{y}, MS_{z}, Q₀, Q₁, Q₂, Q₃, V₁^{f1}, V₁^{f2}, . . . , V₁^{fj}, . . . V₁^{fm}, V₂^{f1}, V₂^{f2}, . . . , V₂^{fj}, . . . , V₂^{fm}, Vᵢ^{f1}, Vᵢ^{f2}, . . . , Vᵢ^{fj}, . . . Vᵢ^{fm}, . . . , Vₙ^{f1}, Vₙ^{f2}, . . . , Vₙ^{fj}, . . . . Vₙ^{fm}] [tₘ₊₁, MS^{x}, MS^{y}, MS^{z}, Q⁰, Q¹, Q², Q³, V₁^{f1}, V₁^{f2}, . . . , V₁^{fj}, . . . V₁^{fm}, V₂^{f1}, V₂^{f2}, . . . , V₂^{fj}, . . . , V₂^{fm}, . . . , Vᵢ^{f1}, Vᵢ^{f2}, . . . , Vᵢ^{fj}, . . . Vᵢ^{fm}, . . . , Vₙ^{f1}, Vₙ^{f2}, . . . , Vₙ^{fj}, . . . Vₙ^{fm}]] assume there are p frames and n electrodes on catheter 120. In an embodiment, generating localization training data 156 may include fixing a reference electrode (say E4) and choosing query electrodes: for p frames of data from n electrodes on catheter 120 one gets p(pn-1) tuples [MS, Vref, Δref_fixed, V query]; and O(p²m) localization training data 156. Alternatively, in some embodiments, generating localization training data 156 may include choosing reference electrode and choosing query electrode: for p frames of data from n electrode on catheter 120 one gets pn (pn-1) tuples [MS, Vref, Δref_fixed, V query]; and O(p²m²) localization training data 156. The model (z,31 ) is trained to predict the A query given MS, V_{ref}, Δ_{ref}, and V_{query}. Therefore, − ([ MS, V_{ref}, Δ_{ref}, V_{query}])→Δ_{query}. In an embodiment, the model may be trained in a supervised formulation if the location of V_{query} is known. Once trained the model may generate inferences using the following data: [tₘ, MSₓ, MS_{y}, MS_{z}, Q₀, Q₁, Q₂, Q₃, V₁^{f1}, V₁^{f2}, . . . , V₁^{fj}, . . . V₁^{fm}, V₂^{f1}, V₂^{f2}, . . . , V₂^{fj}, . . . , V₂^{fm}, . . . , Vᵢ^{f1}, Vᵢ^{f2}, . . . , Vᵢ^{fj}, . . . Vᵢ^{fm}, . . . , Vₙ^{f1}, Vₙ^{f2}, . . . , Vₙ^{fj}, . . . Vₙ^{fm}]. This process formulates n-1 tuples exhausting all the electrodes with respect to a rigidly chosen reference electrode and then predicts, using the trained model. Using the predicted set of query A and magnetic sensor 140, the formulation generates the exact location of the query electrodes in the magnetic field coordinates.

In continued reference to FIG. 1, in some embodiments, localization machine-learning model 152 may be trained treating the rigid electrode as a reference. In such an embodiment, it may be required to have a known location of the reference electrode and a known query electrode. In some embodiments, at least a processor 108 may be further configured to receive shape constraint data 160, wherein shape constraint data 160 relates to relative positions of the one or more electrodes. Additionally, in such an embodiment determining, using localization machine-learning model 152, the location of the one or more query electrodes may further include applying shape constraint data 160 to each of the sets of locations of the plurality of query electrodes to determine a best fit set of locations. As used throughout this disclosure, “shape constraint data” is data related to the shape, size, and/or layout of a given catheter 120. shape constraint data 160 may be integrated with system 100 in accordance with any embodiment of catheter 120 as discussed here within and/or within common use. The following formulation may be utilized to train localization machine-learning model 152 on the rigid electrodes and shape constraint data 160 of a given catheter 120, to estimate the location of the flexible electrodes. Assuming data from catheter 120 is [tₘ, MSₓ, MS_{y}, MS_{z}, Q₀, Q₁, Q₂, Q₃, V₁^{f1}, V₁^{f2}, . . . , V₁^{fj}, . . . V₁^{fm}, V₂^{f1}, V₂^{f2}, . . . , V₂^{fj}, . . . , V₂^{fm}, . . . Vᵢ^{f1}, Vᵢ^{f2}, . . . , Vᵢ^{fj}, . . . Vᵢ^{fm}, . . . , V₁₈^{f1}, V₁₈^{f2}, . . . , V₁₈^{fj}, . . . V₁₈^{fm]}. In an embodiment, there may be 18 electrodes, where 0-15 (G0-G15) are flexible electrodes and 16 (S1) and 17 (S2) are rigid electrodes. A suitable transformation or conversion may be applied by normalization machine-learning model 164 to standardize all voltages across electrode types. Taking S1 as a reference, localization machine-learning model 152 may predict the location of all G1-G15 electrodes: [symbol] ([MS, Vₛ₁, Δₛ₁, V_{Gi}]) →Δ_{Gi} . . . [1]. Taking S2 as a reference, localization machine-learning model 152 may predict the location of all G1-G15 electrodes: [symbol] ([MS, Vₛ₂, Δₛ₂, V_{gi}])→Δ_{Gi} . . . [2].

Taking Gᵢ predicted location from the previous equation [1] as reference, localization machine-learning model 152 may predict the location of S1 and S2: [symbol] ([MS, V_{Gi}, Δ_{Gi}, V_{S1}])→Δ_{S1} . . . [3] and [symbol] ([MS, V_{G2}, Δ_{Gi}, V_{S2}])→Δ_{S2} . . . [4]. Now knowing the location of S1 and S2, localization machine-learning model 152 may use the predicted Δ_{S1} and Δ_{S2} to generate new coordinates for Gᵢ again. Similarly, taking Gᵢ predicted location from the above equation [2] as a reference, localization machine-learning model 152 may predict the location of S1 and S2: [symbol] ([MS, V_{Gi}, Δ_{Gi}, V_{S1}])→Δ_{S1} . . . [5] and [symbol] ([MS, V_{G2}, Δ_{Gi}, V_{S2}])→Δ_{S2} . . . [6]. With the known location of S1 and S2, localization machine-learning model 152 may use the predicted Δ_{S1} and Δ_{S2} to generate new coordinates for Gᵢ again. In total six locations of each Gᵢ are generated. Using the physical catheter 120 constraints, otherwise described above as shape constraint data 160, localization machine-learning model 152 may evaluate the best fit across all permutations possible and select a location set corresponding to minimum cost. Said embodiment may give way to the location of grid electrodes which may be further utilized for training localization machine-learning model 152 and/or prediction directly.

In further reference to FIG. 1, in one or more embodiments normalization training data 168 may be acquired through one or more processes as discussed above in detail. Additionally, In a nonlimiting example, normalization machine-learning model 164 described herein may be consistent with any acquisition of normalization training data 168 and scaling disclosed in U.S. patent application Ser. No. XX/______ (attorney docket number 1518-189USU1), filed on MONTH #, 2024, and entitled “METHODS AND SYSTEMS FOR SCALING LOCATION MODEL DATA” the entirety of which is incorporated herein by reference.

Still referring to FIG. 1, once localization machine-learning model 152 has been trained and/or retrained, system 100 may be configured to input plurality of potential signals 128 and position data 144 in real-time. Input may be manual and/or directly from catheter 120. Then a query electrode location 172 will be determined by localization machine-learning model 152. Query electrode location 172 may be represented through nomenclature and maps, as relative to anatomical landmarks, as spatial coordinates, and/or as visual representations, such as diagrams and schematics. Inputs and its correlated outputs may be used to retrain localization model as described above and in further detail below. Further, query electrode location 172 may be displayed at display device 172. Display device 172 may be any display device as discussed throughout this disclosure, see below for further detail.

Referring now to FIG. 2A, a diagram illustrating an exemplary embodiment of a catheter with electrodes in a grid orientation. As used throughout this disclosure, a “grid orientation” when referring to electrodes, refers to an orientation in which the electrodes are placed in line with one another in a grid-like fashion. This orientation may be useful in detecting a wider range. A grid of electrodes 204a is shown. In an embodiment grid of electrodes 204a may include a plurality of electrodes. Further these electrodes may be flexible electrodes. In one or more embodiments, an individual electrode within grid of electrodes 204a may be 1 mm in length. In such an embodiment, each individual electrode within the grid of electrodes 204a may be spaced 3 mm from each other individual electrode of the grid of electrodes 204a, catheter 200a may additionally include one or more shaft electrodes 208a. Shaft electrodes 208a may be rigid electrodes and located on the shaft of catheter 200a, proximal to grid of electrodes 204a. Furthermore, catheter 200a may include a magnetic sensor 212a. Magnetic sensor 212a may be located on the shaft of catheter 200a, proximal to grid of electrodes 204a and situated between the one or more shaft electrodes 208a. Catheter 200a is an exemplary embodiment of a catheter that may be integrated with system 100.

Referring now to FIG. 2B, a diagram illustrating an exemplary embodiment of a catheter with electrodes in a straight-line orientation. Catheter 200b is yet another exemplary embodiment of a catheter that may be integrated with system 100. Here, catheter 200b includes a contact force sensor 204b, an electrode magnetic sensor 208b, and a braided structure 212b. Contact force sensor 204b may be located behind the distal tip of catheter 200b. Proximal to contact force sensor 204b is where electrode magnetic sensor 208b may be located and is illustrated in FIG. 2B. Further, catheter 200b may integrate a unique braided structure 212b. Braided structure 212b may increase shaft pliability compared to proximal shaft. Additionally, catheter 200b may include 2-2-2 ring spacing for evenly spaced bipole pairs. Lastly, the interior of catheter 200b may include three fiber optic sensing cables. This embodiment, and others not specifically disclosed within may be seamlessly integrated into system 100.

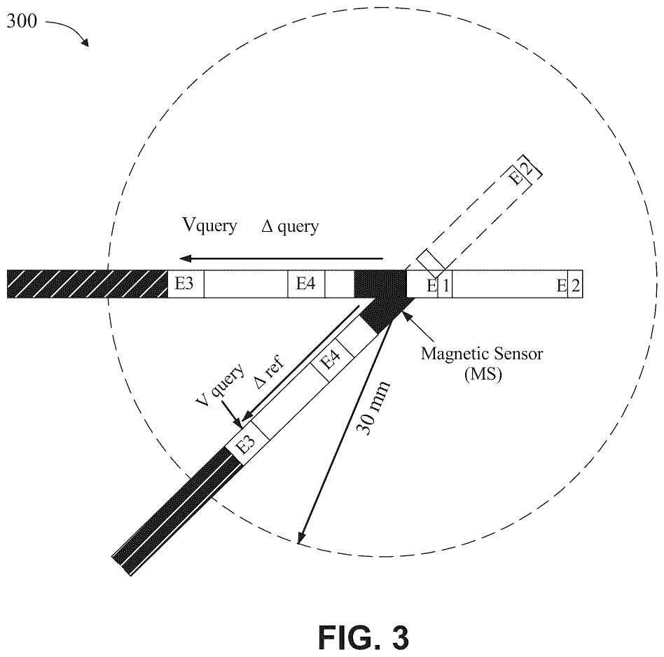

Referring now to FIG. 3 a diagram of a particular implementation of a system for AI-assisted medical device localization 300, specifically illustrating an exemplary embodiment of the at least one or more sensors of a medical device using one or more rigid electrodes as a reference electrode. Illustrated in FIG. 3 are two types of electrodes. Electrodes E1 and E2 on the flexible portion are smaller in size compared to E3 and E4. Further, E1 and E2 are located on the flexible portion of the catheter, meaning there is an ability to bend the catheter in this area. Electrodes E3 and E4 on the rigid portion are larger than E1 and E2 and there is no ability in which to bend the catheter with respect to the lined portion of the stem. A magnetic sensor is embedded in the rigid portion itself. Thus, there are two sets of non-identical electrodes: first set {E1, E2} and a second set {E3, E4}. The electrodes may vary from each other due to their material type, size, shape, and/or electrical characteristics. The voltage read by the electrodes of different types will be different at the same location x, y, z in the field. Therefore, to formulate a generic mapping formulation the voltages must be scaled or otherwise normalized.

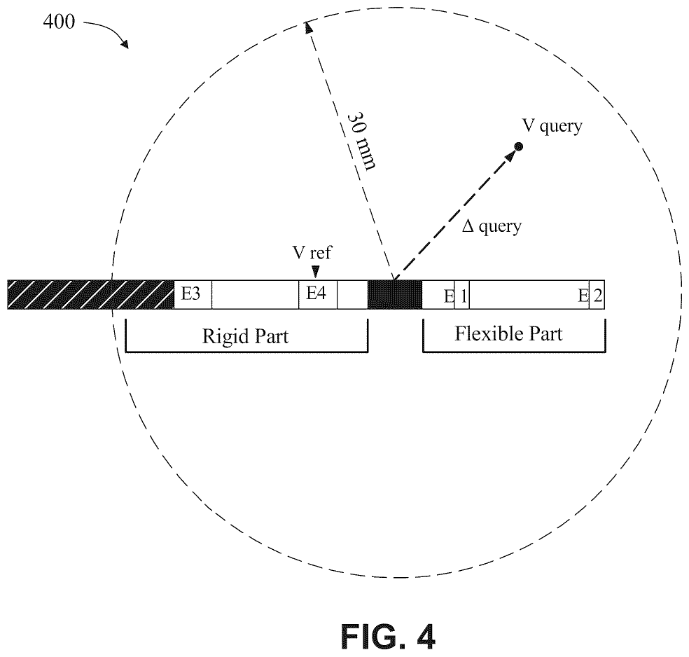

Referring now to FIG. 4, a diagram of a particular implementation of a system for AI-assisted medical device localization 400, specifically illustrating an exemplary embodiment of the at least one or more sensors of a medical device utilizing non-rigid electrodes. Here, rather than rigid electrodes being the reference electrode, flexible electrodes are able to serve as reference electrodes after the training of the localization model.

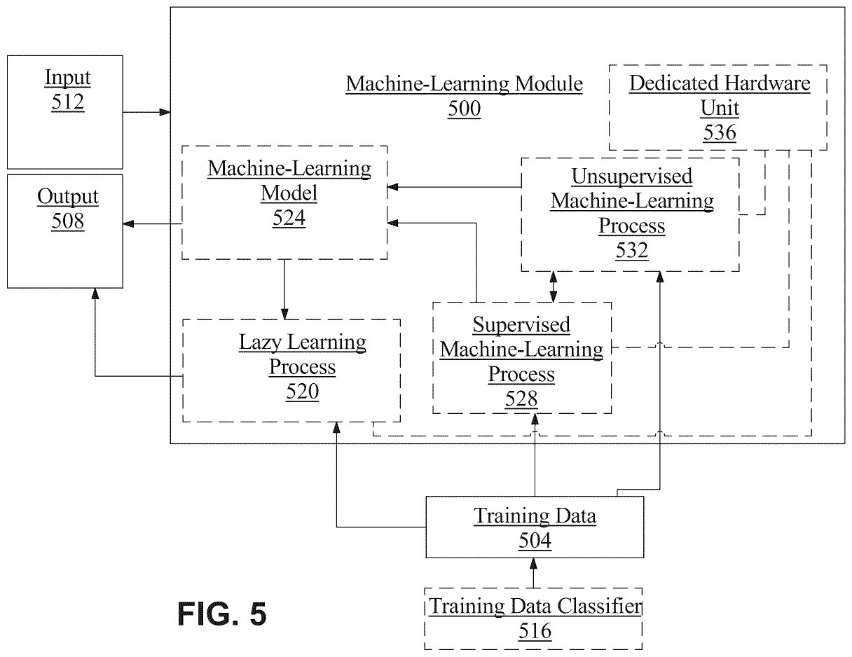

Referring now to FIG. 5, an exemplary embodiment of a machine-learning module 500 that may perform one or more machine-learning processes as described in this disclosure is illustrated. Machine-learning module may perform determinations, classification, and/or analysis steps, methods, processes, or the like as described in this disclosure using machine-learning processes. A “machine-learning process,” as used in this disclosure, is a process that automatedly uses training data 504 to generate an algorithm instantiated in hardware or software logic, data structures, and/or functions that will be performed by a computing device/module to produce outputs 508 given data provided as inputs 512; this is in contrast to a non-machine-learning software program where the commands to be executed are determined in advance by a user and written in a programming language.

Still referring to FIG. 5, “training data,” as used herein, is data containing correlations that a machine-learning process may use to model relationships between two or more categories of data elements. For instance, and without limitation, training data 504 may include a plurality of data entries, also known as “training examples,” each entry representing a set of data elements that were recorded, received, and/or generated together; data elements may be correlated by shared existence in a given data entry, by proximity in a given data entry, or the like. Multiple data entries in training data 504 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 504 according to various correlations; correlations may indicate causative and/or predictive links between categories of data elements, which may be modeled as relationships such as mathematical relationships by machine-learning processes as described in further detail below. Training data 504 may be formatted and/or organized by categories of data elements, for instance by associating data elements with one or more descriptors corresponding to categories of data elements. As a non-limiting example, training data 504 may include data entered in standardized forms by persons or processes, such that entry of a given data element in a given field in a form may be mapped to one or more descriptors of categories. Elements in training data 504 may be linked to descriptors of categories by tags, tokens, or other data elements; for instance, and without limitation, training data 504 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 5, training data 504 may include one or more elements that are not categorized; that is, training data 504 may not be formatted or contain descriptors for some elements of data. Machine-learning algorithms and/or other processes may sort training data 504 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 504 to be made applicable for two or more distinct machine-learning algorithms as described in further detail below. Training data 504 used by machine-learning module 500 may correlate any input data as described in this disclosure to any output data as described in this disclosure. As a non-limiting illustrative example magnetic sensor location data, reference electrode voltage and position data, and query electrode voltage data correlated with query electrode position data.

Further referring to FIG. 5, training data may be filtered, sorted, and/or selected using one or more supervised and/or unsupervised machine-learning processes and/or models as described in further detail below; such models may include without limitation a training data classifier 516. Training data classifier 516 may include a “classifier,” which as used in this disclosure is a machine-learning model as defined below, such as a data structure representing and/or using a mathematical model, neural net, or program generated by a machine-learning algorithm known as a “classification algorithm,” as described in further detail below, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. A distance metric may include any norm, such as, without limitation, a Pythagorean norm. Machine-learning module 500 may generate a classifier using a classification algorithm, defined as a processes whereby a computing device and/or any module and/or component operating thereon derives a classifier from training data 504. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 516 may classify elements of training data to scale or normalize electrical characteristics across non-identical electrode types.

Still referring to FIG. 5, Computing device may be configured to generate a classifier using a Naïve Bayes classification algorithm. Naïve Bayes classification algorithm generates classifiers by assigning class labels to problem instances, represented as vectors of element values. Class labels are drawn from a finite set. Naïve Bayes classification algorithm may include generating a family of algorithms that assume that the value of a particular element is independent of the value of any other element, given a class variable. Naïve Bayes classification algorithm may be based on Bayes Theorem expressed as P(A/B)=P(B/A)P(A)÷P(B), where P(A/B) is the probability of hypothesis A given data B also known as posterior probability; P(B/A) is the probability of data B given that the hypothesis A was true; P(A) is the probability of hypothesis A being true regardless of data also known as prior probability of A; and P(B) is the probability of the data regardless of the hypothesis. A naïve Bayes algorithm may be generated by first transforming training data into a frequency table. Computing device may then calculate a likelihood table by calculating probabilities of different data entries and classification labels. Computing device may utilize a naïve Bayes equation to calculate a posterior probability for each class. A class containing the highest posterior probability is the outcome of prediction. Naïve Bayes classification algorithm may include a gaussian model that follows a normal distribution. Naïve Bayes classification algorithm may include a multinomial model that is used for discrete counts. Naïve Bayes classification algorithm may include a Bernoulli model that may be utilized when vectors are binary.

With continued reference to FIG. 5, Computing device may be configured to generate a classifier using a K-nearest neighbors (KNN) algorithm. A “K-nearest neighbors algorithm” as used in this disclosure, includes a classification method that utilizes feature similarity to analyze how closely out-of-sample-features resemble training data to classify input data to one or more clusters and/or categories of features as represented in training data; this may be performed by representing both training data and input data in vector forms, and using one or more measures of vector similarity to identify classifications within training data, and to determine a classification of input data. K-nearest neighbors algorithm may include specifying a K-value, or a number directing the classifier to select the k most similar entries training data to a given sample, determining the most common classifier of the entries in the database, and classifying the known sample; this may be performed recursively and/or iteratively to generate a classifier that may be used to classify input data as further samples. For instance, an initial set of samples may be performed to cover an initial heuristic and/or “first guess” at an output and/or relationship, which may be seeded, without limitation, using expert input received according to any process as described herein. As a non-limiting example, an initial heuristic may include a ranking of associations between inputs and elements of training data. Heuristic may include selecting some number of highest-ranking associations and/or training data elements.

With continued reference to FIG. 5, generating k-nearest neighbors algorithm may generate a first vector output containing a data entry cluster, generating a second vector output containing an input data, and calculate the distance between the first vector output and the second vector output using any suitable norm such as cosine similarity, Euclidean distance measurement, or the like. Each vector output may be represented, without limitation, as an n-tuple of values, where n is at least two values. Each value of n-tuple of values may represent a measurement or other quantitative value associated with a given category of data, or attribute, examples of which are provided in further detail below; a vector may be represented, without limitation, in n-dimensional space using an axis per category of value represented in n-tuple of values, such that a vector has a geometric direction characterizing the relative quantities of attributes in the n-tuple as compared to each other. Two vectors may be considered equivalent where their directions, and/or the relative quantities of values within each vector as compared to each other, are the same; thus, as a non-limiting example, a vector represented as [5, 10, 15] may be treated as equivalent, for purposes of this disclosure, as a vector represented as [1, 2, 3]. Vectors may be more similar where their directions are more similar, and more different where their directions are more divergent; however, vector similarity may alternatively or additionally be determined using averages of similarities between like attributes, or any other measure of similarity suitable for any n-tuple of values, or aggregation of numerical similarity measures for the purposes of loss functions as described in further detail below. Any vectors as described herein may be scaled, such that each vector represents each attribute along an equivalent scale of values. Each vector may be “normalized,” or divided by a “length” attribute, such as a length attribute l as derived using a Pythagorean norm:

$$l = \sqrt{{\sum }_{i = 0}^{n} {a}_{i}^{2}} ,$$

where aᵢ is attribute number i of the vector. Scaling and/or normalization may function to make vector comparison independent of absolute quantities of attributes, while preserving any dependency on similarity of attributes; this may, for instance, be advantageous where cases represented in training data are represented by different quantities of samples, which may result in proportionally equivalent vectors with divergent values.

With further reference to FIG. 5, training examples for use as training data may be selected from a population of potential examples according to cohorts relevant to an analytical problem to be solved, a classification task, or the like. Alternatively or additionally, training data may be selected to span a set of likely circumstances or inputs for a machine-learning model and/or process to encounter when deployed. For instance, and without limitation, for each category of input data to a machine-learning process or model that may exist in a range of values in a population of phenomena such as images, user data, process data, physical data, or the like, a computing device, processor, and/or machine-learning model may select training examples representing each possible value on such a range and/or a representative sample of values on such a range. Selection of a representative sample may include selection of training examples in proportions matching a statistically determined and/or predicted distribution of such values according to relative frequency, such that, for instance, values encountered more frequently in a population of data so analyzed are represented by more training examples than values that are encountered less frequently. Alternatively or additionally, a set of training examples may be compared to a collection of representative values in a database and/or presented to a user, so that a process can detect, automatically or via user input, one or more values that are not included in the set of training examples. Computing device, processor, and/or module may automatically generate a missing training example; this may be done by receiving and/or retrieving a missing input and/or output value and correlating the missing input and/or output value with a corresponding output and/or input value collocated in a data record with the retrieved value, provided by a user and/or other device, or the like.

Continuing to refer to FIG. 5, computer, processor, and/or module may be configured to preprocess training data. “Preprocessing” training data, as used in this disclosure, is transforming training data from raw form to a format that can be used for training a machine-learning model. Preprocessing may include sanitizing, feature selection, feature scaling, data augmentation and the like.

Still referring to FIG. 5, computer, processor, and/or module may be configured to sanitize training data. “Sanitizing” training data, as used in this disclosure, is a process whereby training examples are removed that interfere with convergence of a machine-learning model and/or process to a useful result. For instance, and without limitation, a training example may include an input and/or output value that is an outlier from typically encountered values, such that a machine-learning algorithm using the training example will be adapted to an unlikely amount as an input and/or output; a value that is more than a threshold number of standard deviations away from an average, mean, or expected value, for instance, may be eliminated. Alternatively or additionally, one or more training examples may be identified as having poor quality data, where “poor quality” is defined as having a signal to noise ratio below a threshold value. Sanitizing may include steps such as removing duplicative or otherwise redundant data, interpolating missing data, correcting data errors, standardizing data, identifying outliers, and the like. In a nonlimiting example, sanitization may include utilizing algorithms for identifying duplicate entries or spell-check algorithms.

As a non-limiting example, and with further reference to FIG. 5, images used to train an image classifier or other machine-learning model and/or process that takes images as inputs or generates images as outputs may be rejected if image quality is below a threshold value. For instance, and without limitation, computing device, processor, and/or module may perform blur detection, and eliminate one or more Blur detection may be performed, as a non-limiting example, by taking Fourier transform, or an approximation such as a Fast Fourier Transform (FFT) of the image and analyzing a distribution of low and high frequencies in the resulting frequency-domain depiction of the image; numbers of high-frequency values below a threshold level may indicate blurriness. As a further non-limiting example, detection of blurriness may be performed by convolving an image, a channel of an image, or the like with a Laplacian kernel; this may generate a numerical score reflecting a number of rapid changes in intensity shown in the image, such that a high score indicates clarity and a low score indicates blurriness. Blurriness detection may be performed using a gradient-based operator, which measures operators based on the gradient or first derivative of an image, based on the hypothesis that rapid changes indicate sharp edges in the image, and thus are indicative of a lower degree of blurriness. Blur detection may be performed using Wavelet-based operator, which takes advantage of the capability of coefficients of the discrete wavelet transform to describe the frequency and spatial content of images. Blur detection may be performed using statistics-based operators take advantage of several image statistics as texture descriptors in order to compute a focus level. Blur detection may be performed by using discrete cosine transform (DCT) coefficients in order to compute a focus level of an image from its frequency content.

Continuing to refer to FIG. 5, computing device, processor, and/or module may be configured to precondition one or more training examples. For instance, and without limitation, where a machine-learning model and/or process has one or more inputs and/or outputs requiring, transmitting, or receiving a certain number of bits, samples, or other units of data, one or more training examples' elements to be used as or compared to inputs and/or outputs may be modified to have such a number of units of data. For instance, a computing device, processor, and/or module may convert a smaller number of units, such as in a low pixel count image, into a desired number of units, for instance by upsampling and interpolating. As a non-limiting example, a low pixel count image may have 100 pixels, however a desired number of pixels may be 128. Processor may interpolate the low pixel count image to convert the 100 pixels into 128 pixels. It should also be noted that one of ordinary skill in the art, upon reading this disclosure, would know the various methods to interpolate a smaller number of data units such as samples, pixels, bits, or the like to a desired number of such units. In some instances, a set of interpolation rules may be trained by sets of highly detailed inputs and/or outputs and corresponding inputs and/or outputs downsampled to smaller numbers of units, and a neural network or other machine-learning model that is trained to predict interpolated pixel values using the training data. As a non-limiting example, a sample input and/or output, such as a sample picture, with sample-expanded data units (e.g., pixels added between the original pixels) may be input to a neural network or machine-learning model and output a pseudo replica sample-picture with dummy values assigned to pixels between the original pixels based on a set of interpolation rules. As a non-limiting example, in the context of an image classifier, a machine-learning model may have a set of interpolation rules trained by sets of highly detailed images and images that have been downsampled to smaller numbers of pixels, and a neural network or other machine-learning model that is trained using those examples to predict interpolated pixel values in a facial picture context. As a result, an input with sample-expanded data units (the ones added between the original data units, with dummy values) may be run through a trained neural network and/or model, which may fill in values to replace the dummy values. Alternatively or additionally, processor, computing device, and/or module may utilize sample expander methods, a low-pass filter, or both. As used in this disclosure, a “low-pass filter” is a filter that passes signals with a frequency lower than a selected cutoff frequency and attenuates signals with frequencies higher than the cutoff frequency. The exact frequency response of the filter depends on the filter design. Computing device, processor, and/or module may use averaging, such as luma or chroma averaging in images, to fill in data units in between original data units.

In some embodiments, and with continued reference to FIG. 5, computing device, processor, and/or module may down-sample elements of a training example to a desired lower number of data elements. As a non-limiting example, a high pixel count image may have 256 pixels, however a desired number of pixels may be 128. Processor may down-sample the high pixel count image to convert the 256 pixels into 128 pixels. In some embodiments, processor may be configured to perform downsampling on data. Downsampling, also known as decimation, may include removing every Nth entry in a sequence of samples, all but every Nth entry, or the like, which is a process known as “compression,” and may be performed, for instance by an N-sample compressor implemented using hardware or software. Anti-aliasing and/or anti-imaging filters, and/or low-pass filters, may be used to clean up side-effects of compression.

Further referring to FIG. 5, feature selection includes narrowing and/or filtering training data to exclude features and/or elements, or training data including such elements, that are not relevant to a purpose for which a trained machine-learning model and/or algorithm is being trained, and/or collection of features and/or elements, or training data including such elements, on the basis of relevance or utility for an intended task or purpose for a trained machine-learning model and/or algorithm is being trained. Feature selection may be implemented, without limitation, using any process described in this disclosure, including without limitation using training data classifiers, exclusion of outliers, or the like.

With continued reference to FIG. 5, feature scaling may include, without limitation, normalization of data entries, which may be accomplished by dividing numerical fields by norms thereof, for instance as performed for vector normalization. Feature scaling may include absolute maximum scaling, wherein each quantitative datum is divided by the maximum absolute value of all quantitative data of a set or subset of quantitative data. Feature scaling may include min-max scaling, in which each value X has a minimum value Xₘᵢₙ in a set or subset of values subtracted therefrom, with the result divided by the range of the values, give maximum value in the set or subset Xₘₐₓ:

$${X}_{new} = \frac{X - {X}_{min}}{{X}_{max} - {X}_{min}} .$$

Feature scaling may include mean normalization, which involves use of a mean value of a set and/or subset of values, Xₘₑₐₙ with maximum and minimum values:

$${X}_{new} = \frac{X - {X}_{mean}}{{X}_{max} - {X}_{min}} .$$

Feature scaling may include standardization, where a difference between X and Xₘₑₐₙ is divided by a standard deviation σ of a set or subset of values:

$${X}_{new} = \frac{X - {X}_{mean}}{σ} .$$

Scaling may be performed using a median value of a set or subset X_{median} and/or interquartile range (IQR), which represents the difference between the 25^{th} percentile value and the 50^{th} percentile value (or closest values thereto by a rounding protocol), such as:

$${X}_{new} = \frac{X - {X}_{median}}{IQR} .$$

Persons skilled in the art, upon reviewing the entirety of this disclosure, will be aware of various alternative or additional approaches that may be used for feature scaling.

Further referring to FIG. 5, computing device, processor, and/or module may be configured to perform one or more processes of data augmentation. “Data augmentation” as used in this disclosure is addition of data to a training set using elements and/or entries already in the dataset. Data augmentation may be accomplished, without limitation, using interpolation, generation of modified copies of existing entries and/or examples, and/or one or more generative AI processes, for instance using deep neural networks and/or generative adversarial networks; generative processes may be referred to alternatively in this context as “data synthesis” and as creating “synthetic data.” Augmentation may include performing one or more transformations on data, such as geometric, color space, affine, brightness, cropping, and/or contrast transformations of images.

Still referring to FIG. 5, machine-learning module 500 may be configured to perform a lazy-learning process 520 and/or protocol, which may alternatively be referred to as a “lazy loading” or “call-when-needed” process and/or protocol, may be a process whereby machine-learning is conducted upon receipt of an input to be converted to an output, by combining the input and training set to derive the algorithm to be used to produce the output on demand. For instance, an initial set of simulations may be performed to cover an initial heuristic and/or “first guess” at an output and/or relationship. As a non-limiting example, an initial heuristic may include a ranking of associations between inputs and elements of training data 504. Heuristic may include selecting some number of highest-ranking associations and/or training data 504 elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail below.

Alternatively or additionally, and with continued reference to FIG. 5, machine-learning processes as described in this disclosure may be used to generate machine-learning models 524. A “machine-learning model,” as used in this disclosure, is a data structure representing and/or instantiating a mathematical and/or algorithmic representation of a relationship between inputs and outputs, as generated using any machine-learning process including without limitation any process as described above, and stored in memory; an input is submitted to a machine-learning model 524 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 524 may be generated by creating an artificial neural network, such as a convolutional neural network comprising an input layer of nodes, one or more intermediate layers, and an output layer of nodes. Connections between nodes may be created via the process of “training” the network, in which elements from a training data 504 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning.

Still referring to FIG. 5, machine-learning algorithms may include at least a supervised machine-learning process 528. At least a supervised machine-learning process 528, as defined herein, include algorithms that receive a training set relating a number of inputs to a number of outputs, and seek to generate one or more data structures representing and/or instantiating one or more mathematical relations relating inputs to outputs, where each of the one or more mathematical relations is optimal according to some criterion specified to the algorithm using some scoring function. For instance, a supervised learning algorithm may include any inputs as described above as inputs, any outputs as described throughout this disclosure as outputs, and a scoring function representing a desired form of relationship to be detected between inputs and outputs; scoring function may, for instance, seek to maximize the probability that a given input and/or combination of elements inputs is associated with a given output to minimize the probability that a given input is not associated with a given output. Scoring function may be expressed as a risk function representing an “expected loss” of an algorithm relating inputs to outputs, where loss is computed as an error function representing a degree to which a prediction generated by the relation is incorrect when compared to a given input-output pair provided in training data 504. Persons skilled in the art, upon reviewing the entirety of this disclosure, will be aware of various possible variations of at least a supervised machine-learning process 528 that may be used to determine relation between inputs and outputs. Supervised machine-learning processes may include classification algorithms as defined above.

With further reference to FIG. 5, training a supervised machine-learning process may include, without limitation, iteratively updating coefficients, biases, weights based on an error function, expected loss, and/or risk function. For instance, an output generated by a supervised machine-learning model using an input example in a training example may be compared to an output example from the training example; an error function may be generated based on the comparison, which may include any error function suitable for use with any machine-learning algorithm described in this disclosure, including a square of a difference between one or more sets of compared values or the like. Such an error function may be used in turn to update one or more weights, biases, coefficients, or other parameters of a machine-learning model through any suitable process including without limitation gradient descent processes, least-squares processes, and/or other processes described in this disclosure. This may be done iteratively and/or recursively to gradually tune such weights, biases, coefficients, or other parameters. Updating may be performed, in neural networks, using one or more back-propagation algorithms. Iterative and/or recursive updates to weights, biases, coefficients, or other parameters as described above may be performed until currently available training data is exhausted and/or until a convergence test is passed, where a “convergence test” is a test for a condition selected as indicating that a model and/or weights, biases, coefficients, or other parameters thereof has reached a degree of accuracy. A convergence test may, for instance, compare a difference between two or more successive errors or error function values, where differences below a threshold amount may be taken to indicate convergence. Alternatively or additionally, one or more errors and/or error function values evaluated in training iterations may be compared to a threshold.

Still referring to FIG. 5, a computing device, processor, and/or module may be configured to perform method, method step, sequence of method steps and/or algorithm described in reference to this figure, in any order and with any degree of repetition. For instance, a computing device, processor, and/or module may be configured to perform a single step, sequence and/or algorithm repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. A computing device, processor, and/or module may perform any step, sequence of steps, or algorithm in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Further referring to FIG. 5, machine-learning processes may include at least an unsupervised machine-learning processes 532. An unsupervised machine-learning process, as used herein, is a process that derives inferences in datasets without regard to labels; as a result, an unsupervised machine-learning process may be free to discover any structure, relationship, and/or correlation provided in the data. Unsupervised processes 532 may not require a response variable; unsupervised processes 532 may be used to find interesting patterns and/or inferences between variables, to determine a degree of correlation between two or more variables, or the like.

Still referring to FIG. 5, machine-learning module 500 may be designed and configured to create a machine-learning model 524 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the clastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 5, machine-learning algorithms may include, without limitation, linear discriminant analysis. Machine-learning algorithm may include quadratic discriminant analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include various forms of latent space regularization such as variational regularization. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized trees, AdaBoost, gradient tree boosting, and/or voting classifier methods. Machine-learning algorithms may include neural net algorithms, including convolutional neural net processes.

Still referring to FIG. 5, a machine-learning model and/or process may be deployed or instantiated by incorporation into a program, apparatus, system and/or module. For instance, and without limitation, a machine-learning model, neural network, and/or some or all parameters thereof may be stored and/or deployed in any memory or circuitry. Parameters such as coefficients, weights, and/or biases may be stored as circuit-based constants, such as arrays of wires and/or binary inputs and/or outputs set at logic “1” and “0” voltage levels in a logic circuit to represent a number according to any suitable encoding system including twos complement or the like or may be stored in any volatile and/or non-volatile memory. Similarly, mathematical operations and input and/or output of data to or from models, neural network layers, or the like may be instantiated in hardware circuitry and/or in the form of instructions in firmware, machine-code such as binary operation code instructions, assembly language, or any higher-order programming language. Any technology for hardware and/or software instantiation of memory, instructions, data structures, and/or algorithms may be used to instantiate a machine-learning process and/or model, including without limitation any combination of production and/or configuration of non-reconfigurable hardware elements, circuits, and/or modules such as without limitation ASICs, production and/or configuration of reconfigurable hardware elements, circuits, and/or modules such as without limitation FPGAs, production and/or of non-reconfigurable and/or configuration non-rewritable memory elements, circuits, and/or modules such as without limitation non-rewritable ROM, production and/or configuration of reconfigurable and/or rewritable memory elements, circuits, and/or modules such as without limitation rewritable ROM or other memory technology described in this disclosure, and/or production and/or configuration of any computing device and/or component thereof as described in this disclosure. Such deployed and/or instantiated machine-learning model and/or algorithm may receive inputs from any other process, module, and/or component described in this disclosure, and produce outputs to any other process, module, and/or component described in this disclosure.

Continuing to refer to FIG. 5, any process of training, retraining, deployment, and/or instantiation of any machine-learning model and/or algorithm may be performed and/or repeated after an initial deployment and/or instantiation to correct, refine, and/or improve the machine-learning model and/or algorithm. Such retraining, deployment, and/or instantiation may be performed as a periodic or regular process, such as retraining, deployment, and/or instantiation at regular elapsed time periods, after some measure of volume such as a number of bytes or other measures of data processed, a number of uses or performances of processes described in this disclosure, or the like, and/or according to a software, firmware, or other update schedule. Alternatively or additionally, retraining, deployment, and/or instantiation may be event-based, and may be triggered, without limitation, by user inputs indicating sub-optimal or otherwise problematic performance and/or by automated field testing and/or auditing processes, which may compare outputs of machine-learning models and/or algorithms, and/or errors and/or error functions thereof, to any thresholds, convergence tests, or the like, and/or may compare outputs of processes described herein to similar thresholds, convergence tests or the like. Event-based retraining, deployment, and/or instantiation may alternatively or additionally be triggered by receipt and/or generation of one or more new training examples; a number of new training examples may be compared to a preconfigured threshold, where exceeding the preconfigured threshold may trigger retraining, deployment, and/or instantiation.

Still referring to FIG. 5, retraining and/or additional training may be performed using any process for training described above, using any currently or previously deployed version of a machine-learning model and/or algorithm as a starting point. Training data for retraining may be collected, preconditioned, sorted, classified, sanitized or otherwise processed according to any process described in this disclosure. Training data may include, without limitation, training examples including inputs and correlated outputs used, received, and/or generated from any version of any system, module, machine-learning model or algorithm, apparatus, and/or method described in this disclosure; such examples may be modified and/or labeled according to user feedback or other processes to indicate desired results, and/or may have actual or measured results from a process being modeled and/or predicted by system, module, machine-learning model or algorithm, apparatus, and/or method as “desired” results to be compared to outputs for training processes as described above.

Redeployment may be performed using any reconfiguring and/or rewriting of reconfigurable and/or rewritable circuit and/or memory elements; alternatively, redeployment may be performed by production of new hardware and/or software components, circuits, instructions, or the like, which may be added to and/or may replace existing hardware and/or software components, circuits, instructions, or the like.

Further referring to FIG. 5, one or more processes or algorithms described above may be performed by at least a dedicated hardware unit 536. A “dedicated hardware unit,” for the purposes of this figure, is a hardware component, circuit, or the like, aside from a principal control circuit and/or processor performing method steps as described in this disclosure, that is specifically designated or selected to perform one or more specific tasks and/or processes described in reference to this figure, such as without limitation preconditioning and/or sanitization of training data and/or training a machine-learning algorithm and/or model. A dedicated hardware unit 536 may include, without limitation, a hardware unit that can perform iterative or massed calculations, such as matrix-based calculations to update or tune parameters, weights, coefficients, and/or biases of machine-learning models and/or neural networks, efficiently using pipelining, parallel processing, or the like; such a hardware unit may be optimized for such processes by, for instance, including dedicated circuitry for matrix and/or signal processing operations that includes, e.g., multiple arithmetic and/or logical circuit units such as multipliers and/or adders that can act simultaneously and/or in parallel or the like. Such dedicated hardware units 536 may include, without limitation, graphical processing units (GPUs), dedicated signal processing modules, FPGA or other reconfigurable hardware that has been configured to instantiate parallel processing units for one or more specific tasks, or the like, A computing device, processor, apparatus, or module may be configured to instruct one or more dedicated hardware units 536 to perform one or more operations described herein, such as evaluation of model and/or algorithm outputs, one-time or iterative updates to parameters, coefficients, weights, and/or biases, and/or any other operations such as vector and/or matrix operations as described in this disclosure.

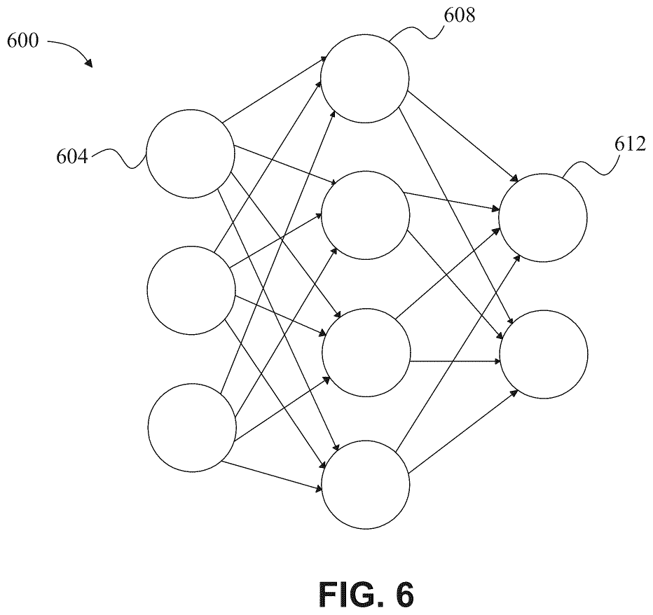

Referring now to FIG. 6, an exemplary embodiment of neural network 600 is illustrated. A neural network 600 also known as an artificial neural network, is a network of “nodes,” or data structures having one or more inputs, one or more outputs, and a function determining outputs based on inputs. Such nodes may be organized in a network, such as without limitation a convolutional neural network, including an input layer of nodes 604, one or more intermediate layers 608, and an output layer of nodes 612. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. Connections may run solely from input nodes toward output nodes in a “feed-forward” network, or may feed outputs of one layer back to inputs of the same or a different layer in a “recurrent network.” As a further non-limiting example, a neural network may include a convolutional neural network comprising an input layer of nodes, one or more intermediate layers, and an output layer of nodes. A “convolutional neural network,” as used in this disclosure, is a neural network in which at least one hidden layer is a convolutional layer that convolves inputs to that layer with a subset of inputs known as a “kernel,” along with one or more additional layers such as pooling layers, fully connected layers, and the like.

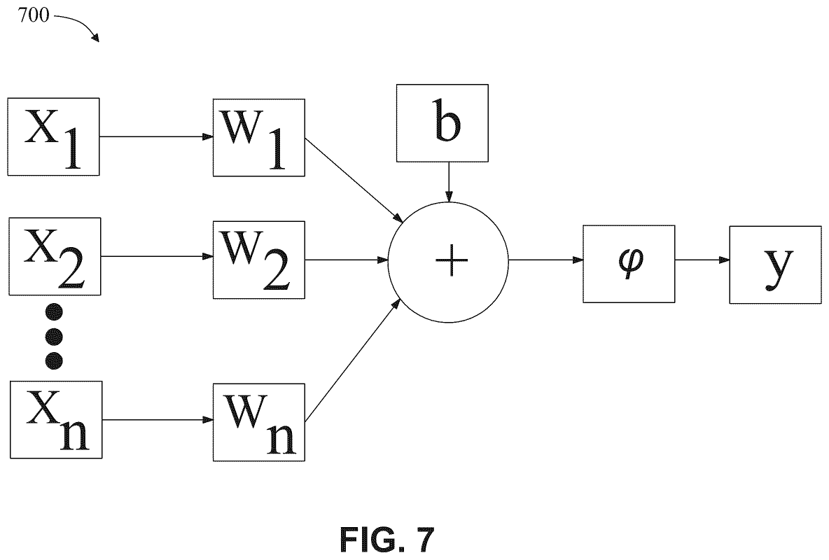

Referring now to FIG. 7, an exemplary embodiment of a node 700 of a neural network is illustrated. A node may include, without limitation a plurality of inputs xᵢ that may receive numerical values from inputs to a neural network containing the node and/or from other nodes. Node may perform one or more activation functions to produce its output given one or more inputs, such as without limitation computing a binary step function comparing an input to a threshold value and outputting either a logic 1 or logic 0 output or something equivalent, a linear activation function whereby an output is directly proportional to the input, and/or a non-linear activation function, wherein the output is not proportional to the input. Non-linear activation functions may include, without limitation, a sigmoid function of the form

$$f ( x ) = \frac{1}{1 - {e}^{- x}}$$

given input x, a tanh (hyperbolic tangent) function, of the form

$$\frac{{e}^{x} - {e}^{- x}}{{e}^{x} + {e}^{- x}} ,$$

a tanh derivative function such as f(x)=tanh²(x), a rectified linear unit function such as f(x)=max(0, x), a “leaky” and/or “parametric” rectified linear unit function such as f(x)=max(ax, x) for some a, an exponential linear units function such as

$$f ( x ) = \{ \begin{matrix}x for x \geq 0 \\ α ( {e}^{x} - 1 ) for x < 0\end{matrix}$$

for some value of α (this function may be replaced and/or weighted by its own derivative in some embodiments), a softmax function such as

$$f ( {x}_{i} ) = \frac{{e}^{x}}{{\sum }_{i} {x}_{i}}$$

where the inputs to an instant layer are xᵢ, a swish function such as f(x)=x*sigmoid(x), a Gaussian error linear unit function such as f(x)=α(1+tanh(\sqrt{2/π}(x+bx^{r}))) for some values of a, b, and r, and/or a scaled exponential linear unit function such as

$$f ( x ) = λ \{ \begin{matrix}α ( {e}^{x} - 1 ) for x < 0 \\ x for x \geq 0\end{matrix} .$$

Fundamentally, there is no limit to the nature of functions of inputs xᵢ that may be used as activation functions. As a non-limiting and illustrative example, node may perform a weighted sum of inputs using weights wᵢ that are multiplied by respective inputs xᵢ. Additionally or alternatively, a bias b may be added to the weighted sum of the inputs such that an offset is added to each unit in the neural network layer that is independent of the input to the layer. The weighted sum may then be input into a function φ, which may generate one or more outputs y. Weight wᵢ applied to an input xᵢ may indicate whether the input is “excitatory,” indicating that it has strong influence on the one or more outputs y, for instance by the corresponding weight having a large numerical value, and/or a “inhibitory,” indicating it has a weak effect influence on the one more inputs y, for instance by the corresponding weight having a small numerical value. The values of weights wᵢ may be determined by training a neural network using training data, which may be performed using any suitable process as described above.

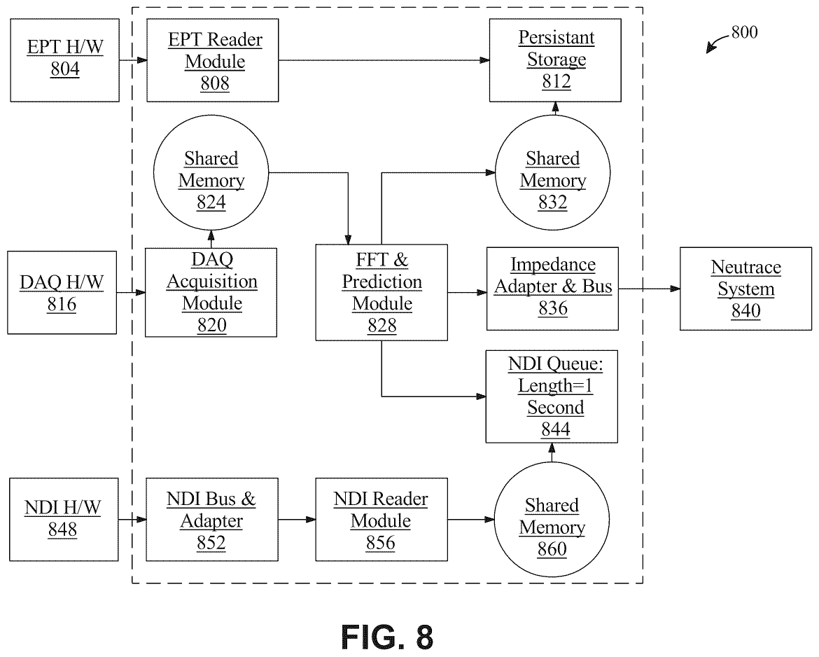

Now referring to FIG. 8, illustrated is an exemplary software flow diagram of system 800 for various modules. EPT H/W 804 is an electrophysiology tracer, which is configured to measure and record intracardiac and ECG voltage measurements. EPT H/W 804 may have dedicated channels for surface ECG patches, and catheter signals, which may come through a change control board (CCB) box after splitting them. EPT H/W 804 may work at a sampling rate of 1 kHz and thus suitable for signals within the 0-500 Hz range. The rate of the intracardiac signal being measured by EPT H/W 804 may be 1000 samples per second across all channels (i.e., surface ECG and EMG signals). The internal circuitry of EPT H/W 804 may provide provision for filtering and amplifications per channel. Within system 800 EPT H/W may input data as discussed above, into EPT Reader Module 808. EPT Reader Module 808 may include a model architecture as described throughout this disclosure, for example and without limitation, such as model 900 discussed below. EPT Reader Module 808 may output logs, or otherwise write logs to disk and send said logs to persistent storage 812. As used here, and throughout this disclosure, “persistent storage” is a data storage device that can retain data even when the device is powered off or disconnected from a system.

Still referring to FIG. 8, DAQ H/W 816 is a data acquisition system. Signals from one or more catheter pins may be split and fed to EPT H/W 804 as well as to DAQ H/W 816. An exemplary DAQ H/W 816 is a system built by National Instruments (NI). DAQ H/W 816 may connect to the processing computer via a LAN interface. DAQ H/W may input collected data into DAQ acquisition module 816. Within DAQ acquisition module 816 there may be any one and/or combination of three types of modules: (1) patch voltage generation module with six channels and +/−10 V range; (2) patch voltage measurement module with +/−10 V, 12 bit resolution, and 8 channels; and (3) catheter voltage measurement module with a max range of +/−1V with 16-bit resolution, giving a sensitivity of 15 microvolts. The acquisition rate for all the measurement modules is 30 KHz, thus providing a time resolution of 33 microseconds. From DAQ acquisition module 816 outputs may be sent to shared memory 824. For example, and without limitation buffer data may be written ever 100 ms. From shared memory 824 buffer data may be read by FFT and prediction module 828. FFT stands for Fast Fourier Transform, which is a mathematical algorithm that converts time-domain data into a frequency-domain representation. From FFT and prediction module 828 three potential paths exist as discussed below. As further disclosure regarding FFT and prediction module 828, 100-millisecond window of signal measurements at every 100 ms are acquired. Thus, the read frequency provides 10windows of 100 ms per second. Windowing of the signal allows for continuous signal measurement of a varying signal and can be estimated over a sufficient window to counter any noise-related erroneous values. To estimate the voltage emerging due to the subjected electrical field, FFT is used as a way to measure the voltage response of every electrode within an acquired window. One window of the data acquired via DAQ H/W 816 may present as such: [tDAQ, V buffer window]. Where, t_{DAQ} is mid time of the acquisition window; V _{buffer window} is an R^{nxz} matrix where n is the number of electrodes being tracked DAQ, and z is 2000 for 30 KHz sampling rate and 100 ms window. In one or more embodiments, a conditioning step may occur for each electrode. In such an embodiment, the signal conditioning step may have a mean subtraction, zero padding, and a Hanning window before FFT. After FFT a peak detection may occur, wherein the amplitudes of the normalized voltage are taken at the patch frequencies. For the i^{th} electrode, we get Vᵢ^{f1}, Vᵢ^{f2}, . . . , Vᵢ^{fj}, . . . . Vᵢ^{fm} where f1, . . . , fm are m excitation frequencies. The DAQ data after signal conditioning may present as such: [t_{DAQ}, V₁^{f1}, V₁^{f1}, . . . , V₁^{fj}, . . . V₁^{fm}, V₂^{f1}, V₂^{f2}, . . . , V₂^{fj}, . . . , V₂^{fm}, Vᵢ^{f1}, Vᵢ^{f2}, . . . , Vᵢ^{fj}, . . . Vᵢ^{fm}, . . . , Vₙ^{f1}, Vₙ^{f2}, . . . , Vₙ^{fj}, . . . Vₙ^{fm}].

Continuing to reference FIG. 8, from FFT and prediction module 828 buffer data and FFT data along with matched timestamps may be written on shared memory 832. Further shared memory 832 may write buffer data and FFT data to HDF5 on storage at persistent storage 812. Alternatively from FFT and prediction module 828 predictions may be sent via network to impedance adapter and BUS 836, wherein impedance adapter and BUS 836 may communicate said predictions via Rabbit MQ to Neutrace system 840. Additionally, and alternatively, from FFT and prediction module 828 buffer data may be read for the closest NDI record by timestamp matching at NDI queue: length=1 second 844.

Further referencing FIG. 8, Neutrace system 840 may be configured to run on a centralized rendering system, wherein all data streams are sent to said centralized rendering system. Neutrace system 840 is an interface where a user, such as a surgeon, may see the map involved in real-time along with the catheter electrodes being rendered to facilitate navigation. Apart form the geometry building, Neutrace system 840 may also be responsible for the point acquisition gating based on a user-defined template rhythm on the ECG and/or EGM signal.

In further reference to FIG. 8, NDI H/W 848 is a software program configured to facilitate magnetic field generation. NDI H/W 848 may operate in coordination with NDI field generator hardware, a software driver, and am adaptor code running on a computer system. NDI H/W 848 may employ a sophisticated method creating a cylindrical magnetic field, useful for high-precision tracking in medical procedures. NDI H/W 848 may utilize an electromagnetic transmitter equipped with multiple carefully arranged coils. Said coils may generate a magnetic field by passing an electrical current through them, producing a stable, predictable field within a cylindrical volume centered around the transmitter. The cylindrical shape of this field is designed to ensure consistent tracking accuracy within the operational area. Sensors may be embedded in medical instruments to detect variations in the magnetic field's strength and direction. NDI H/W 848 may process these variations to calculate the precise position and orientation of the sensors in real-time. The cylindrical magnetic field is especially suited for procedures where maintaining precise control over instrument placement is crucial. For example, and without limitation, such as in catheter navigation and other minimally invasive interventions. NDI H/W 848 may possess measurement accuracy within a few millimeters. Positional accuracy of magnetic tracking systems, such as NDI H/W 848 may range from +/−1 mm to +/−2 mm. This means that the tracked position of the sensor is within this error range from its true position. The angular accuracy, which measures the precision of orientation tracking may fall within +/−1 degree to +/−2 degrees. In one or more embodiments, NDI H/W 848 may be configured to track one or more magnetic sensors and/or catheters simultaneously. NDI H/W 848 may be configured to track eight sensors and/or catheters using two signal interface units (SIU) and one signal control unit (SCU). The throughput of NDI H/W 848 is 33 samples per second, assuming a velocity range of 0-8 cm per second of the catheter and/or sensor tip. In an embodiment wherein the velocity is higher, NDI H/W 848 may provide a catheter missing warning/A typical sample of the magnetic data may be presented as such: [tₘ, MSₓ¹, MS_{y}¹, MS_{z}¹, Q₀¹, Q₁¹, Q₂¹, Q₃¹, . . . MSₓ^{k}, MS_{y}^{k}, MS_{z}^{k}, Q₀^{k}, Q₁^{k}, Q₂^{k}, Q₃^{k}, . . . MSₓ^{p}, MS_{y}^{p}, MS_{z}^{p}, Q₀^{p}, Q₁^{p}, Q₂^{p}, Q₃^{p}]. Wherein tₘ is the time of the reading and/or acquisition; MSx^{k} is magnetic sensor x location of the catheter (mm) for the k^{th} sensor and/or catheter; MSy^{k} is magnetic sensor y location of the catheter (mm) for the k^{th} sensor and/or catheter; MSz^{k} is magnetic sensor z location of the catheter (mm) for the k^{th} sensor and/or catheter; Q₀^{k}, Q₁^{k}, Q₂^{k}, Q₃^{k}, are quaternion coefficient providing orientation for the k^{th} sensor and/or catheter; and p is the total number of catheters and/or sensors.

Continuing to reference FIG. 8, NDI H/W 848 may input data as discussed above into NDI BUS and adapter 852. From NDI BUS and adapter 852 data may be read every 100 ms via a transmission control protocol (TCP) at NDI reader module 856. Further, from NDI reader module 856 parsed NDI records may be written at shared memory 860. Which may further store recent one second NDI records at NDI queue: length=1 second 844.

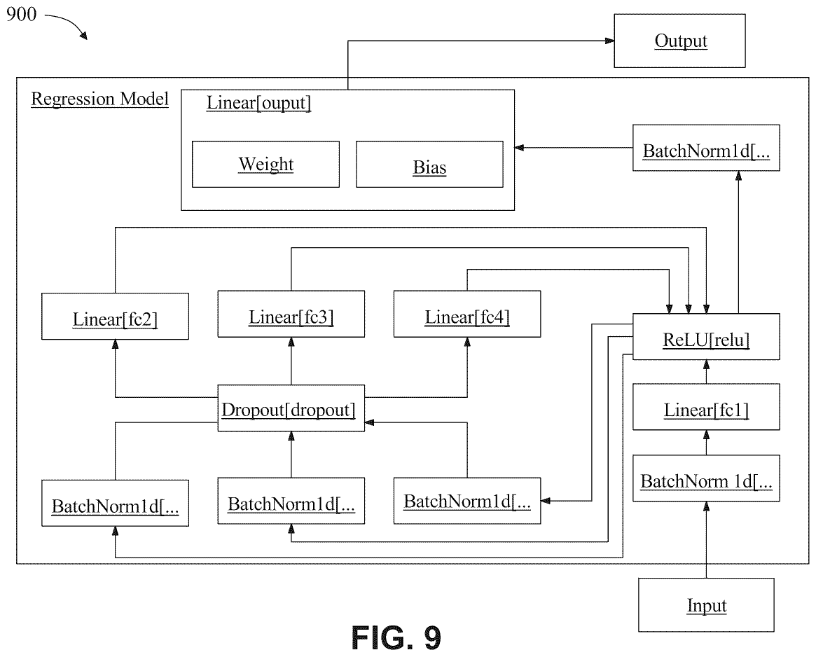

Now referring to FIG. 9, illustrated is an exemplary embodiment of model architecture 900. Model architecture 900 is a regression model including 3,867 total number of parameters. Exemplary inputs may be any input as described throughout this disclosure. Further, exemplary outputs may be any output as described throughout this disclosure.

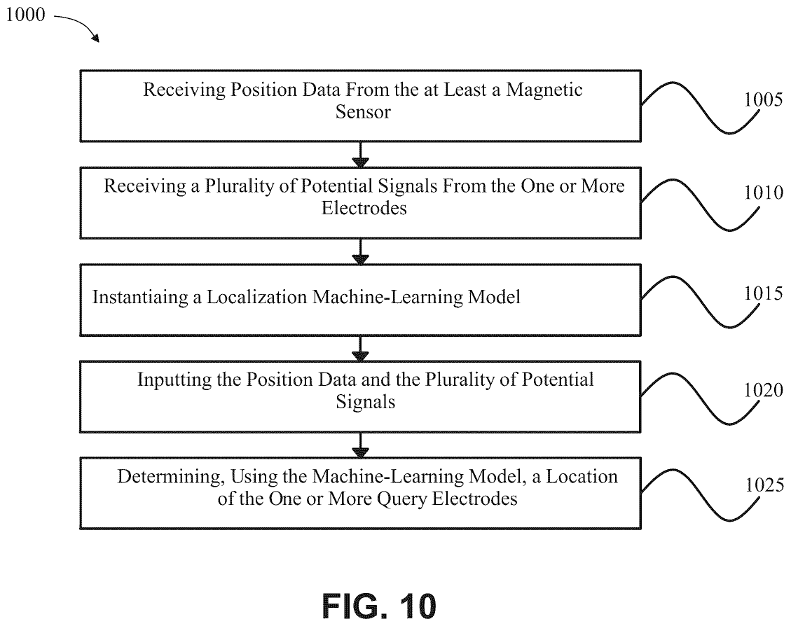

Now referring to FIG. 10, illustrated is a flow diagram of a particular implementation of various steps of a method of AI assisted medical device localization. In an embodiment, a method for AI assisted medical device localization 1000 includes receiving position data from the at least a magnetic sensor 1005, receiving a plurality of potential signals from the one or more electrodes 1010, instantiating a localization machine-learning model 1015, inputting the position data and the plurality of potential signals 1020, and determining, using the localization machine-learning model a location of the one or more query electrodes 1025.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

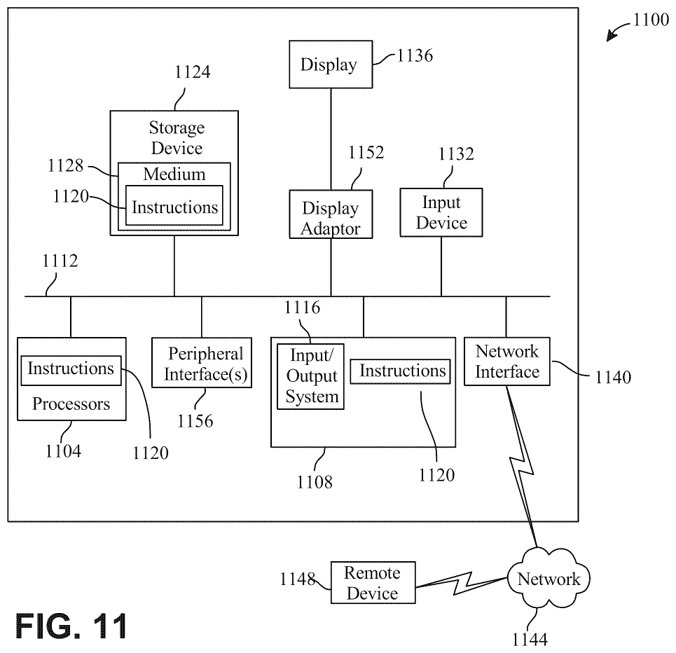

FIG. 11 shows a diagrammatic representation of one embodiment of a computing device in the exemplary form of a computer system 1100 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1100 includes a processor 1104 and a memory 1108 that communicate with each other, and with other components, via a bus 1112. Bus 1112 may include any of several types of bus structures including, but not limited to, a memory bus, a memory controller, a peripheral bus, a local bus, and any combinations thereof, using any of a variety of bus architectures.

Processor 1104 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1104 may be organized according to Von Neumann and/or Harvard architecture as a non-limiting example. Processor 1104 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), system on module (SOM), and/or system on a chip (SoC).

Memory 1108 may include various components (e.g., machine-readable media) including, but not limited to, a random-access memory component, a read only component, and any combinations thereof. In one example, a basic input/output system 1116 (BIOS), including basic routines that help to transfer information between elements within computer system 1100, such as during start-up, may be stored in memory 1108. Memory 1108 may also include (e.g., stored on one or more machine-readable media) instructions (e.g., software) 1120 embodying any one or more of the aspects and/or methodologies of the present disclosure. In another example, memory 1108 may further include any number of program modules including, but not limited to, an operating system, one or more application programs, other program modules, program data, and any combinations thereof.

Computer system 1100 may also include a storage device 1124. Examples of a storage device (e.g., storage device 1124) include, but are not limited to, a hard disk drive, a magnetic disk drive, an optical disc drive in combination with an optical medium, a solid-state memory device, and any combinations thereof. Storage device 1124 may be connected to bus 1112 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1124 (or one or more components thereof) may be removably interfaced with computer system 1100 (e.g., via an external port connector (not shown)). Particularly, storage device 1124 and an associated machine-readable medium 1128 may provide nonvolatile and/or volatile storage of machine-readable instructions, data structures, program modules, and/or other data for computer system 1100. In one example, software 1120 may reside, completely or partially, within machine-readable medium 1128. In another example, software 1120 may reside, completely or partially, within processor 1104.

Computer system 1100 may also include an input device 1132. In one example, a user of computer system 1100 may enter commands and/or other information into computer system 1100 via input device 1132. Examples of an input device 1132 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1132 may be interfaced to bus 1112 via any of a variety of interfaces (not shown) including, but not limited to, a serial interface, a parallel interface, a game port, a USB interface, a FIREWIRE interface, a direct interface to bus 1112, and any combinations thereof. Input device 1132 may include a touch screen interface that may be a part of or separate from display 1136, discussed further below. Input device 1132 may be utilized as a user selection device for selecting one or more graphical representations in a graphical interface as described above.

A user may also input commands and/or other information to computer system 1100 via storage device 1124 (e.g., a removable disk drive, a flash drive, etc.) and/or network interface device 1140. A network interface device, such as network interface device 1140, may be utilized for connecting computer system 1100 to one or more of a variety of networks, such as network 1144, and one or more remote devices 1148 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1144, may employ a wired and/or a wireless mode of communication. In general, any network topology may be used. Information (e.g., data, software 1120, etc.) may be communicated to and/or from computer system 1100 via network interface device 1140.

Computer system 1100 may further include a video display adapter 1152 for communicating a displayable image to a display device, such as display device 1136. Examples of a display device include, but are not limited to, a liquid crystal display (LCD), a cathode ray tube (CRT), a plasma display, a light emitting diode (LED) display, and any combinations thereof. Display adapter 1152 and display device 1136 may be utilized in combination with processor 1104 to provide graphical representations of aspects of the present disclosure. In addition to a display device, computer system 1100 may include one or more other peripheral output devices including, but not limited to, an audio speaker, a printer, and any combinations thereof. Such peripheral output devices may be connected to bus 1112 via a peripheral interface 1156. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A system for artificial intelligence assisted medical device localization, wherein the system comprises:
at least a processor;
a catheter communicatively connected to the at least a processor, wherein the catheter further comprises:
one or more electrodes configured to detect a plurality of potential signals, including a first reference electrode, a second reference electrode, and at least a query electrode flexibly connected to the first reference electrode and the second reference electrode and configured to detect at least a query electrode datum, wherein the one or more electrodes are part of an electrical impedance-based positioning system; and
at least a magnetic sensor configured to detect position data, wherein the at least a magnetic sensor is part of a magnetic field-based positioning system; and
a memory communicatively connected to the at least a processor, wherein the memory contains instructions configuring the at least a processor to:
receive position data from the at least a magnetic sensor of the magnetic field-based positioning system;
receive a plurality of potential signals from the one or more electrodes of the electrical impedance-based positioning system, wherein the plurality of potential signals comprises:
at least a reference electrode datum; and
the at least a query electrode datum;
instantiate a localization machine-learning model;
input the position data of the magnetic field-based positioning system and the plurality of potential signals of the electrical impedance-based positioning system into the localization machine-learning model; and
determine, using the localization machine-learning model, a location of the at least a query electrode by:
predicting a first predicted location of the at least a query electrode using at least a first reference datum of a first reference electrode as a reference;
predicting a second predicted location of the at least a query electrode using at least a second reference electrode datum of the second reference electrode as a reference;
predicting locations of the first and second reference electrodes based on the first and second predicted locations of the at least a query electrode derived from the first and second reference electrode datums of the first and second reference electrodes;
determining the location of the at least a query electrode from an electrical potential of the at least a query electrode datum, received from the one or more electrodes of the electrical impedance-based positioning system, in combination with the position data, received from the at least a magnetic sensor of the magnetic field-based positioning system, and the predicted locations of the first and second reference electrodes; and
utilizing a feedback mechanism, by using previous results of the localization machine-learning model, across prediction rounds to update relative position values and coordinates for the location of the at least a query electrode.

2. The system of claim 1, wherein the memory further configures the at least a processor to display, at a display device, the location of the at least a query electrode.

3. The system of claim 1, wherein the one or more electrodes comprise:
one or more rigid electrodes, wherein the one or more rigid electrodes detect the at least a reference electrode datum; and
one or more flexible electrodes, wherein the one or more flexible electrodes detect the at least a query electrode datum.

4. The system of claim 3, wherein the one or more electrodes are positioned in a grid orientation.

5. The system of claim 1, wherein the one or more electrodes are positioned in a straight-line orientation.

6. The system of claim 1, wherein determining, using the localization machine-learning model, the location of the at least a query electrode further comprises iteratively determining sets of locations of a plurality of query electrodes using a plurality of reference electrodes.

7. The system of claim 6, wherein iteratively determining sets of locations of a plurality of query electrodes using a plurality of reference electrodes comprises training the localization machine-learning model on exemplary magnetic sensor location data, exemplary reference electrode voltage and position data, and exemplary query electrode voltage data correlated with query electrode position data.

8. The system of claim 7, wherein:
the memory contains instructions further configuring the at least a processor to receive shape constraint data, wherein the shape constraint data relates to relative positions of at least a query electrode relative the first reference electrode and the second reference electrode; and
determining, using the localization machine-learning model, the location of the at least a query electrode further comprises applying the shape constraint data to each of the first and second predicted location of the at least a query electrode to determine a best fit set of locations.

9. The system of claim 1, wherein:
the one or more electrodes comprise one or more non-identical electrodes; and
the memory contains instructions further configuring the at least a processor to normalize the plurality of potential signals of the one or more non-identical electrodes, wherein normalizing the plurality of potential signals of the one or more non-identical electrodes comprises:
instantiating a normalization machine-learning model;
receiving, at the at least a processor, a first type of electrode data having a first type of electrode characteristics;
receiving, at the at least a processor, a second type of electrode data having a second type of electrode characteristics; and
converting, using the normalization machine-learning model, the first type of electrode data having a first type of electrode characteristics into the second type of electrode data having a second type of electrode characteristics.

10. The system of claim 9, wherein instantiating a normalization machine-learning model comprises:
generating a training dataset, wherein the training dataset comprises exemplary voltage pairs that are measured using the one or more non-identical electrodes at a same location correlated with exemplary scaled voltages;
training the normalization machine-learning model using the training dataset; and
determining, using the normalization machine-learning model, scaled voltages of the one or more non-identical electrodes.

11. A method for artificial intelligence assisted medical device localization, wherein the method comprises:
receiving position data from at least a magnetic sensor of a catheter, wherein the at least a magnetic sensor is part of a magnetic field-based positioning system;
receiving a plurality of potential signals from one or more electrodes of the catheter, wherein the catheter comprises a first reference electrode, a second reference electrode, and at least a query electrode flexibly connected to the first reference electrode and the second reference electrode, wherein the one or more electrodes are part of an electrical impedance-based positioning system, wherein the plurality of potential signals comprises:
at least a reference electrode datum; and
at least a query electrode datum;
instantiating a localization machine-learning model;
inputting the position data of the magnetic field-based positioning system and the plurality of potential signals of the electrical impedance-based positioning system into the localization machine-learning model; and
determining, using the localization machine-learning model, a location of the at least a query electrode by:
predicting a first predicted location of the at least a query electrode using at least a first reference datum of a first reference electrode as a reference;
predicting a second predicted location of the at least a query electrode using at least a second reference electrode datum of the second reference electrode as a reference;
predicting locations of the first and second reference electrodes based on the first and second predicted location of the at least a query electrode derived from the first and second reference electrode datums of the first and second reference electrodes;
determining the location of the at least a query electrode from an electrical potential of the at least a query electrode datum, received from the one or more electrodes of the electrical impedance-based positioning system, in combination with the position data, received from the at least a magnetic sensor of the magnetic field-based positioning system, and the predicted locations of the first and second reference electrodes; and
utilizing a feedback mechanism, by using previous results of the localization machine-learning model, across prediction rounds to update relative position values and coordinates for the location of the at least a query electrode.

12. The method of claim 11, wherein the method further comprises displaying, at a display device, the location of the at least a query electrode.

13. The method of claim 11, wherein the one or more electrodes of the catheter comprise:
one or more rigid electrodes, wherein the one or more rigid electrodes detect at least a reference electrode datum; and
one or more flexible electrodes, wherein the one or more flexible electrodes detect at least a query electrode datum.

14. The method of claim 13, wherein the one or more electrodes of the catheter are positioned in a grid orientation.

15. The method of claim 11, wherein the one or more electrodes of the catheter are positioned in a straight-line orientation.

16. The method of claim 11, wherein determining, using the localization machine-learning model, the location of the at least a query electrode further comprises iteratively determining sets of locations of a plurality of query electrodes using a plurality of reference electrodes.

17. The method of claim 16, wherein iteratively determining sets of locations of a plurality of query electrodes using a plurality of reference electrodes comprises training the localization machine-learning model on exemplary magnetic sensor location data, exemplary reference electrode voltage and position data, and exemplary query electrode voltage data correlated with query electrode position data.

18. The method of claim 17, wherein:
the method further comprises receiving shape constraint data, wherein the shape constraint data relates to relative positions of the at least a query electrode relative to the first reference electrode and the second reference electrode; and
determining, using the localization machine-learning model, the location of the at least a query electrode further comprises applying the shape constraint data to each of the first and second predicted locations of the at least a query electrode to determine a best fit set of locations.

19. The method of claim 11, wherein the one or more electrodes of the catheter are non-identical, and the method further comprises normalizing the plurality of potential signals of the one or more non-identical electrodes, wherein normalizing the plurality of potential signals of the one or more non-identical electrodes comprises:
instantiating a normalization machine-learning model;
receiving a first type of electrode data having a first type of electrode characteristics;
receiving a second type of electrode data having a second type of electrode characteristics; and
converting, using the normalization machine-learning model, the first type of electrode data having a first type of electrode characteristics into the second type of electrode data having a second type of electrode characteristics.

20. The method of claim 19, wherein instantiating a normalization machine-learning model comprises:
generating a training dataset, wherein the training dataset comprises exemplary voltage pairs that are measured using the one or more non-identical electrodes at a same location correlated with exemplary scaled voltages;
training the normalization machine-learning model using the training dataset; and
determining, using the normalization machine-learning model, scaled voltages of the one or more non-identical electrodes.
